(19) United States
(12) Patent Application Publication
(10) Pub. No.: US 2007/0117784 A1
(43) Pub. Date: May 24, 2007

(54) Treatment of hyperproliferative diseases with anthraquinones

(75) Inventors: Jeffrey L. Cleland, San Carlos, CA (US); Alvin Wong, San Francisco, CA (US); Alshad S. Lalani, San Francisco, CA (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Novacea, Inc.

(21) Appl. No.: 11/520,034

(22) Filed: Sep. 13, 2006

Related U.S. Application Data
(63) Continuation-in-part of application No. PCT/US06/07452, filed on Mar. 3, 2006.
(60) Provisional application No. 60/658,371, filed on Mar. 4, 2005.

(51) Int. Cl. A61K 31/55; A61K 31/445; A61K 31/40; A61K 31/13; A61K 31/397

(52) U.S. Cl. 514/183; 514/210.01; 514/212.01; 514/319; 514/408; 514/644

(57) ABSTRACT

The invention relates to anthraquinone compounds having activity for treating hyperproliferative disorders. Further, the invention relates to methods of using the compounds, alone or in combination with one or more other active agents or treatments, to treat hyperproliferative disorders.

### BACKGROUND OF THE INVENTION

1. Field of the Invention

The present invention relates to compounds having activity for treating hyperproliferative disorders. Further, the invention relates to methods of using the compounds, alone or in combination with one or more other active agents or treatments, to treat hyperproliferative disorders.

2. Related Art

One in every four deaths in the United States is due to cancer, and cancer is the second leading cause of death. U.S. Cancer Statistics Working Group; United States Cancer Statistics: 1999-2001 Incidence, Atlanta (Ga.): Department of Health and Human Services, Centers for Disease Control and Prevention, and National Cancer Institute (2004). The National Cancer Institute reports that almost 10 million Americans have a history of invasive cancer, while the American Cancer Society estimates that in the year 2004, over 1.3 million Americans will receive a diagnosis of invasive cancer with over a half million cases resulting in death. American Cancer Society, Cancer Facts & Figures 2004. These statistics exclude the 1 million cases of basal and squamous cell skin cancers that are expected to be diagnosed in the United States.

Cancers are classified based on the organ and cell tissue from which the cancer originates, including: (i) carcinomas (most common kind of cancer which originates in epithelial tissues, the layers of cells covering the body's surface or lining internal organs and various glands); (ii) leukemias (origination in the blood-forming tissues, including bone marrow, lymph nodes and the spleen); (iii) lymphomas (originates in the cells of the lymph system); (iv) melanomas (originates in the pigment cells located among the epithelial cells of the skin); and (v) sarcomas (originates in the connective tissues of the body, such as bones, muscles and blood vessels). (See Molecular Biology of the Cell: Third Edition, “Cancer,” Chapter 24, pp. 1255-1294, B. Alberts et al., (eds.), Garland Publishing, Inc., New York (1994); and Stedman's Pocket Medical Dictionary; Williams and Wilkins, Baltimore (1987)). Within these broad cancer classifications, there are over one hundred cancer subclassifications, such as breast, lung, pancreatic, colon, and prostate cancer, to name a few.

Cancer cells develop as a result of damage to a cell's DNA (i.e., altered DNA sequence or altered expression pattern) from exposure to various chemical agents, radiation, viruses, or when some not-yet-fully-understood internal, cellular signaling event occurs. Most of the time when a cell's DNA becomes damaged, the cell either dies or is able to repair the DNA. However, for cancer cells, the damaged DNA is not repaired and the cell continues to divide, exhibiting modified cell physiology and function.

Neoplasms, or tumors, are masses of cells that result from an aberrant, accelerated rate of growth (i.e., hyperproliferative cell growth). As long as the tumor cells remain confined to a single mass, the tumor is considered to be benign. However, a cancerous tumor has the ability to invade other tissues and is termed malignant. In general, cancer cells are defined by two heritable properties: the cells and their progeny 1) reproduce in defiance of normal restraints, and 2) invade and colonize the territories of other cells.

Cancerous tumors are comprised of a highly complex vasculature and differentiated tissue. A large majority of cancerous tumors have hypoxic components, which are relatively resistant to standard anti-cancer treatment, including radiotherapy and chemotherapy. Brown, Cancer Res. 59:5863 (1999); and Kunz, M. et al., Mol. Cancer 2:1 (2003). Thomlinson and Gray presented the first anatomical model of a human tumor that describes a 100 to 150 μm thick hypoxic layer of tissue located between the blood vessels and necrotic tumor tissues.

Research has shown that the hypoxic tissues within a number of cancerous tumors promote the progression of the cancer by an array of complex mechanisms. See, Brown., supra, and Kunz et al., supra. Among these are activation of certain signal transduction pathways and gene regulatory mechanisms, induction of selection processes for gene mutations, tumor cell apoptosis and tumor angiogenesis. Most of these mechanisms contribute to tumor progression. Therefore, tissue hypoxia has been regarded as a central factor for tumor aggressiveness and metastasis. Therapies that target hypoxic tissues within a tumor would certainly provide improved treatments to patients suffering from tumor-related cancers and/or disorders.

In addition to cancer, there exist a number of hyperproliferative diseases and/or disorders that are associated with the onset of hypoxia in a given tissue. For example, Shweiki et al. explain that inadequate oxygen levels often lead to neovascularization in order to compensate for the needs of the hypoxic tissue. Neovascularization is mediated by expression of certain growth factors, such as vascular endothelial growth factor (VEGF). Shweiki et al., Nature 359:843 (1992). However, when certain tissues or growth factors are either directly or indirectly upregulated in response to hypoxia without sufficient feedback mechanisms for controlling tissue expression, various diseases and/or disorders may ensue (i.e., by hypoxia-aggravated hyperproliferation). By way of example, hypoxia-aggravated hyperproliferative diseases and/or disorders having over-expressed levels of VEGF include ocular angiogenic diseases, such as age-related macular degeneration and diabetic retinopathy, rheumatoid arthritis, as well as cirrhosis of the liver. See Frank, Ophthalmic Res. 29:341 (1997); Ishibashi et al., Graefe's Archive Clin. Exp. Ophthamol. 235:159 (1997); Corpechot et al., Hepatology 35:1010 (2002). In addition to those diseases characterized prominently by hyperproliferation and hypoxia there is a group of disorders characterized by ischemic or hypoxic injury followed by a proliferative response aimed at repair of the tissue damaged by hypoxia and cell death. The two most common disorders are acute myocardial infarction and acute cerebral infarction or stroke.

U.S. Pat. No. 5,132,327 describes a group of anthraquinone prodrug compounds having the following structure: 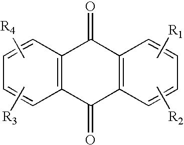
in which R₁, R₂, R₃ and R₄ are each separately selected from the group consisting of hydrogen, X, NH-A-NHR and NH-A-N(O)R′R″ wherein X is hydroxy, halogeno, amino, C₁₋₄ alkoxy or C₂₋₈ alkanoyloxy, A is a C₂₋₄ alkylene group with a chain length between NH and NHR or N(O)R′R″ of at least 2 carbon atoms and R, R′ and R″ are each separately selected from the group consisting of C₁₋₄ alkyl groups and C₂₋₄ hydroxyalkyl and C₂₋₄ dihydroxyalkyl groups in which the carbon atom attached to the nitrogen atom does not carry a hydroxy group and no carbon atom is substituted by two hydroxy groups, or R′ and R″ together are a C₂₋₆ alkylene group which with the nitrogen atom to which R′ and R″ are attached forms a heterocyclic group having 3 to 7 atoms in the ring, but with the proviso that at least one of R₁ to R₄ is a group NH-A-N(O)R′R″, the compound optionally being in the form of a physiologically acceptable salt. These compounds are described as being useful in the treatment of cancer.

Among the compounds disclosed in U.S. Pat. No. 5,132,327 is the compound AQ4N (1,4-bis{[2-(dimethylamino)ethyl]amino}-5,8-dihydroxyanthracene-9,10-dione bis-N-oxide.

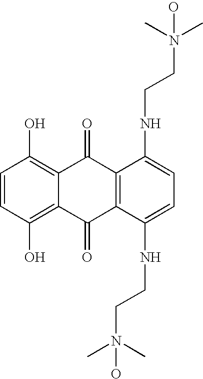

AQ4N has been shown to have potent anti-hyperproliferative activity and to enhance the antitumor effects of radiation and conventional chemotherapeutic agents. Patterson, Drug Metab. Rev. 34:581 (2002). For many tumor cells, AQ4N is not intrinsically cytotoxic; in hypoxic tumors it is converted to the cytotoxic compound AQ4 (1,4-bis{[2-(dimethylamino)ethyl]amino}-5,8-dihydroxyanthracene-9,10-dione). Among the activities associated with AQ4 are intercalation into DNA and inhibition of topoisomerase II activity.

### BRIEF SUMMARY OF THE INVENTION

The present invention is related to compositions and methods for treating hyperproliferative disorders, such as cancer. One aspect of the invention is drawn to methods of treating, ameliorating, or preventing hyperproliferative disease in a subject comprising administering to said subject a therapeutically effective amount of a compound having Formula I: 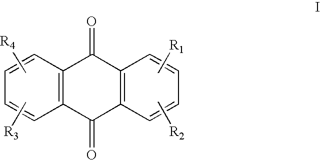
or a pharmaceutically acceptable salt or prodrug thereof, wherein:
R₁, R₂, R₃ and R₄ are independently hydrogen, hydroxy, halo, amino, C₁₋₄ alkoxy, C₂₋₈ alkanoyloxy, NH-A-NHR, or NH-A-N(O)R′R″;
A is a C₂₋₄ alkylene group with a chain length between NH and NHR or N(O)R′R″ of at least 2 carbon atoms; and
R, R′ and R″ are independently C₁₋₄ alkyl, C₂₋₄ hydroxyalkyl, or C₂₋₄ dihydroxyalkyl in which the carbon atom attached to the nitrogen atom does not carry a hydroxy group and no carbon atom is substituted by two hydroxy groups; or
R′ and R″ together are a C₂₋₆ alkylene group which with the nitrogen atom to which R′ and R″ are attached forms a heterocyclic group having 3 to 7 atoms in the ring;
with the proviso that at least one of R₁ to R₄ is NH-A-N(O)R′R″.

Another aspect of the invention is a method of reducing or preventing metastasis of a cancer in an animal comprising administering to an animal in need thereof a therapeutically effective amount of a compound of Formula I.

A further aspect of the invention is a method of improving the efficacy of the cytotoxic response, time to progression, or the overall survival rate of an animal receiving one or more chemotherapeutic agents and/or one or more radiotherapeutic agents/treatments comprising administering to an animal in need thereof a therapeutically effective amount of compound of Formula I in combination with the one or more chemotherapeutic agents, and/or one or more radiotherapeutic agents/treatments.

In one embodiment of the invention, the compound of Formula I is AQ4N.

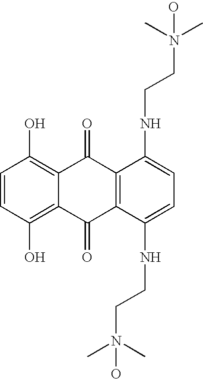

An additional aspect of the present invention is a method for treating, ameliorating, or preventing hyperproliferative disorders in an animal comprising administering to the animal a therapeutically effective amount of a compound having Formula I in combination with one or more active agents or treatments, for example, chemotherapeutic agents or radiotherapeutic agents/treatments.

In preferred embodiments of the invention, the one or more chemotherapeutic agents can be any chemotherapeutic agent which is used, has been used, or is known to be useful for the treatment of hyperproliferative disorders.

In preferred embodiments of the invention, the one or more radiotherapeutic agents or treatments can be external-beam radiation therapy, brachytherapy, thermotherapy, radiosurgery, charged-particle radiotherapy, neutron radiotherapy, photodynamic therapy, or radionuclide therapy.

In one embodiment of the invention, the compound having Formula I can be administered prior to, during, and/or beyond administration of the one or more chemotherapeutic agents or radiotherapeutic agents or treatments. In another embodiment of the invention, the method of administering a compound having Formula I in combination with one or more chemotherapeutic agents or radiotherapeutic agents or treatments is repeated more than once.

The combination of a compound having Formula I and one or more chemotherapeutic agents or radiotherapeutic agents or treatments of the present invention will have additive potency or an additive therapeutic effect. The invention also encompasses synergistic combinations where the therapeutic efficacy is greater than additive. Preferably, such combinations will reduce or avoid unwanted or adverse effects. In certain embodiments, the combination therapies encompassed by the invention will provide an improved overall therapy relative to administration of a compound having Formula I or any chemotherapeutic agent or radiotherapeutic agent or treatment alone. In certain embodiments, doses of existing or experimental chemotherapeutic agents or radiotherapeutic agents or treatments will be reduced or administered less frequently which will increase patient compliance, thereby improving therapy and reducing unwanted or adverse effects.

Further, the methods of the invention will be useful not only with previously untreated patients but also will be useful in the treatment of patients partially or completely refractory to current standard and/or experimental cancer therapies, including but not limited to radiotherapies, chemotherapies, and/or surgery. In a preferred embodiment, the invention will provide therapeutic methods for the treatment or amelioration of hyperproliferative disorders that have been shown to be or may be refractory or non-responsive to other therapies.

While not wishing to be bound by any theory, it is believed that some of the N-oxide compounds of the invention will function as prodrugs with greatly diminished cytotoxicity. It is believed that these N-oxide compounds will be activated under hypoxic conditions within the target tissues (i.e., reduced at the nitrogen atom), followed by intercalation between the base pairs in the host cell DNA. Other N-oxide compound of the invention may have intrinsic cytotoxic activity. It is contemplated that the targets of the compounds for facilitating cell toxicity include DNA, helicases, microtubules, protein kinase C, and topoisomerase I and II. Since a number of pathological tissues have significant hypoxic components which promote hyperproliferation, it is believed that this portion of tissue will be preferentially targeted.

### BRIEF DESCRIPTION OF THE DRAWINGS/FIGURES

FIG. 1 shows the effect of different doses of AQ4N on a P388 chronic lymphocytic leukemia mouse model.

FIG. 2 shows a comparison of the effect of AQ4N, mitoxantrone, and carmustine on a P388 chronic lymphocytic leukemia mouse model.

FIG. 3 shows the effect of different doses of AQ4N on a P388 chronic lymphocytic leukemia mouse model in terms of survival time.

FIG. 4 shows the reproducibility of the effect of different doses of AQ4N on a P388 chronic lymphocytic leukemia mouse model.

FIG. 5 shows the effect of different doses of AQ4N on a L1210 acute lymphocytic leukemia mouse model.

FIG. 6 shows a comparison of the effect of AQ4N, mitoxantrone, and carmustine on a L1210 acute lymphocytic leukemia mouse model.

FIG. 7 shows the effect of different doses of AQ4N on a L1210 acute lymphocytic leukemia mouse model in terms of survival time.

FIG. 8 shows the reproducibility of the effect of different doses of AQ4N on a L1210 acute lymphocytic leukemia mouse model.

FIG. 9 shows the effect of different doses of AQ4N on a Namalwa human lymphoma mouse model.

FIG. 10 shows the effect of different doses of AQ4N on a HT-29 colon cancer mouse model.

FIG. 11 shows the effect of different doses of AQ4N on a HT-29 colon cancer mouse model.

FIG. 12 shows the effect of different doses of AQ4N alone and in combination with irinotecan on a HT-29 colon cancer mouse model.

FIG. 13 shows the effect of different doses of AQ4N alone and in combination with irinotecan on a HT-29 colon cancer mouse model.

FIG. 14 shows the distribution of radiolabeled AQ4N after administration to a mouse.

FIG. 15 shows the distribution of radiolabeled AQ4N after administration to a mouse.

FIG. 16 shows that AQ4N treatment significant delays tumor growth progression in the sc Panc-1 pancreatic adenocarcinoma model. Human Panc-1 tumor fragments were implanted sc into nude mice and AQ4N or gemcitabine treatment (at the indicated dose and schedules) was initiated when tumors reached approximately 100 mm³ in size. Mice were actively sacrificed as a cancer death when their tumors reached 1200 mm³. Shown are Kaplan-Meier plots summarizing the percentage of the animals remaining in the study as a function of time following tumor inoculation. Treatment outcome was determined from TGD, defined as the increase in median TTE in a treatment group as compared to the control group.

FIG. 17 shows the effect of different doses of AQ4N on a BXPC-3 pancreatic cancer mouse model.

FIG. 18 shows the effect of different doses of AQ4N on a BXPC-3 pancreatic cancer mouse model.

FIG. 19 shows the effect of different doses of AQ4N alone and in combination with gemcitabine on a BXPC-3 pancreatic cancer mouse model.

FIG. 20 shows Significant anti-tumor activity by AQ4N treatment in sc and ot pancreatic BxPC-3 xenograft models. A) Inhibition of sc implanted BxPC-3 tumors in nude mice. AQ4N or gemcitabine treatment, using the dose/schedule regimens indicated in the legend, was initiated when tumors reached approximately 60-80 mm³ in size. Shown are the mean (±SEM) tumor volumes measured from treatment and control groups following tumor challenge. The study was terminated when tumors from the vehicle-treated group reached an average of approximately 2 grams (day 27) and the % TGI was calculated as defined in the text. B) Shown are Kaplan-Meier plots demonstrating prolongation of survival by AQ4N in mice bearing ot implanted BxPC-3 tumors. AQ4N or gemcitabine treatment was initiated on day 14 following direct injection of tumor cells into the pancreas parenchyma. Animals were actively sacrificed when they became moribund from excessive tumor burden.

FIG. 21 shows Tumor and plasma pharmacokinetics of AQ4N, AQ4M and AQ4 in BxPC-3-tumor bearing mice. Following a single administration of 20, 60, 120 or 240 mg/kg AQ4N, primary BxPC-3 tumors or plasma samples were collected at 2, 8 and 24 hrs and subject to HPLC/MS/MS for quantitative analysis. Shown are the mean (±SD) plasma and tumor concentrations of AQ4N, AQ4M and AQ4 plotted as a function of time post-treatment (left panels) or vs. treatment dose (right panels). (A) Shown are the plasma concentrations of AQ4N plotted vs. AQ4N treatment dose or time. The bioreduced metabolites, AQ4M and AQ4, were undetectable in the plasma at any of the treatment doses or time points investigated. (B) Shown are the concentrations of AQ4N in the tumor and plotted vs. treatment dose or time. (C) Shown are the concentrations of the intermediate AQ4M metabolite measured in the tumor and plotted as a function of AQ4N treatment dose or time. (D) Shown are the concentrations of the cytotoxic AQ4 metabolite measured in pancreatic tumors and plotted against the input treatment dose or time.

FIG. 22 shows Effects of AQ4N treatment on liver metastases following orthotopic BxPC-3 tumor implantation. A) Livers were resected on day 36 post-tumor challenge, paraffin embedded and stained with H&E. Shown are two representative H&E-stained liver sections from control-treated (top), 60 mg/kg AQ4N-treated (middle) and 120 mg/kg AQ4N-treated 120 mg/kg (bottom) groups. Magnification 100×. B) AQ4N decreases the incidence and invasiveness of metastasis BxPC-3 lesions in the liver (n=5/group). To compute metastasis index, 5 histological sections from each liver were analyzed by microscopy and scored for the mean (±SEM) percentage of area occupied by the invading BxPC-3 tumor lesion in comparison to the total liver tissue area analyzed. Evaluation of blinded samples was performed by two independent operators. Statistical analysis was done using the non-parametric Mann-Whitney U-test.

FIG. 23 shows the additive and synergistic cytotoxic effects of AQ4 (FIG. 23A) or AQ4N (FIG. 23B) treatment in combination with 48 hr treatment with temozolomide to M059K glioblastoma cell lines in vitro. Shown are isobolograms where the 50% inhibitory concentrations (IC₅₀) of each of the test agent were calculated relative to untreated controls for each combination treatment where possible following a 10-day colony formation assay. The calculated IC₅₀ points on the dotted line are indicative of additive cytotoxic effects resulting from the combinatorial treatment and points following below the dotted line are indicative of synergistic cytotoxic interactions following the combinatorial treatment between the two test agents.

FIG. 24 shows the additive and synergistic cytotoxic effects of 48 hr combined treatment of AQ4 (FIG. 24A) or AQ4N (FIG. 24B) with temozolomide and radiation in M059K glioblastoma cell lines in vitro. M059K cells were treated with AQ4 (FIG. 24A) or AQ4N (FIG. 24B) together with temozolomide for 48 hr. 24 hr following treatment, cells were also subject to 1.5 Gy radiation. Shown are isobolograms where the 50% inhibitory concentrations (IC₅₀) of each of the test agent were calculated relative to untreated controls for each combination treatment where possible following a 10-day colony formation assay. The calculated IC₅₀ points on the dotted line are indicative of additive cytotoxic effects, points following below the dotted line are indicative of synergistic cytotoxic interactions, and points above the dotted line are indicative of antagonist interactions resulting from the combinatorial treatment.

### DETAILED DESCRIPTION OF THE INVENTION

One aspect of the invention is drawn to methods of treating, ameliorating, or preventing hyperproliferative disease in a subject comprising administering to said subject a therapeutically effective amount of a compound having Formula I: 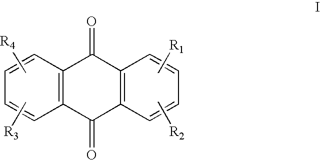
or a pharmaceutically acceptable salt or prodrug thereof, wherein:
R₁, R₂, R₃ and R₄ are independently hydrogen, hydroxy, halo, amino, C₁₋₄ alkoxy, C₂₋₈ alkanoyloxy, NH-A-NHR, or NH-A-N(O)R′R″;
A is a C₂₋₄ alkylene group with a chain length between NH and NHR or N(O)R′R″ of at least 2 carbon atoms; and
R, R′ and R″ are independently C₁₋₄ alkyl, C₂₋₄ hydroxyalkyl, or C₂₋₄ dihydroxyalkyl in which the carbon atom attached to the nitrogen atom does not carry a hydroxy group and no carbon atom is substituted by two hydroxy groups; or
R′ and R″ together are a C₂₋₆ alkylene group which with the nitrogen atom to which R′ and R″ are attached forms a heterocyclic group having 3 to 7 atoms in the ring;
with the proviso that at least one of R₁ to R₄ is NH-A-N(O)R′R″.

Useful alkyl groups include straight-chained or branched C₁₋₁₀ alkyl groups, especially methyl, ethyl, propyl, isopropyl, t-butyl, sec-butyl, 3-pentyl, adamantyl, norbornyl, and 3-hexyl groups.

Useful halo or halogen groups include fluorine, chlorine, bromine and iodine.

Useful alkoxy groups include oxygen substituted by one of the C₁₋₁₀ alkyl groups mentioned above, especially methoxy and ethoxy.

Useful alkanoyloxy groups include acyloxy substituted by one of the C₁₋₁₀ alkyl groups mentioned above, especially acetyl and propionyl.

Useful heterocyclic groups include tetrahydrofuranyl, pyranyl, piperidinyl, piperizinyl, pyrrolidinyl, imidazolidinyl, imidazolinyl, indolinyl, isoindolinyl, quinuclidinyl, morpholinyl, isochromanyl, chromanyl, pyrazolidinyl, pyrazolinyl, tetronoyl and tetramoyl groups.

According to another aspect of the invention, a therapeutically effective amount of a compound having Formula I, or a pharmaceutically acceptable salt thereof, and at least one other active agent is provided in the form of a pharmaceutical composition having at least one pharmaceutically acceptable carrier. In certain instances, the at least one other active agent is a chemotherapeutic agent (including an active vitamin D compound). Compounds having Formula I may be formulated in a single formulation with the other active agent(s), or formulated independently.

According to one aspect of the invention, methods for treating, ameliorating, or preventing hyperproliferative disorders are provided, wherein a therapeutically effective amount of a compound having Formula I, or a pharmaceutically acceptable salt thereof, is administered to an animal in need thereof. In certain aspects of the invention, the hyperproliferative disorder is cancer. In one embodiment, the cancer is a solid tumor. In another embodiment, the cancer is selected from the group consisting of colon cancer, brain cancer, glioma, multiple myeloma, head and neck cancer (except for esophageal cancer), hepatocellular cancer, melanoma, ovarian cancer, cervical cancer, renal cancer, and non-small cell lung cancer.

A further aspect of the invention relates to methods for treating, ameliorating, or preventing a hyperproliferative disorder comprising administering a therapeutically effective amount of a compound having Formula I, or a pharmaceutically acceptable salt thereof, in combination with at least one other active agent or treatment to a patient in need thereof. In certain embodiments, combinations of a compound having Formula I with a chemotherapeutic agent are administered. In one embodiment, the chemotherapeutic agent is selected from gemcitabine and irinotecan.

Hyperproliferative disorders which can be treated with the compounds having Formula I include any hypoxia-aggravated hyperproliferative disease and/or disorder, such as any number of cancers. Generally, such cancers include, without limitation, cancers of the bladder, brain, breast, cervix, colon, endometrium, esophagus, head and neck, kidney, larynx, liver, lung, oral cavity, ovaries, pancreas, prostate, skin, stomach, and testis. Certain of these cancers may be more specifically referred to as acute and chronic lymphocytic leukemia, acute granulocytic leukemia, adrenal cortex carcinoma, bladder carcinoma, breast carcinoma, cervical carcinoma, cervical hyperplasia, choriocarcinoma, chronic granulocytic leukemia, chronic lymphocytic leukemia, colon carcinoma, endometrial carcinoma, esophageal carcinoma, essential thrombocytosis, genitourinary carcinoma, hairy cell leukemia, head and neck carcinoma, Hodgkin's disease, Kaposi's sarcoma, lung carcinoma, lymphoma, malignant carcinoid carcinoma, malignant hypercalcemia, malignant melanoma, malignant pancreatic insulinoma, medullary thyroid carcinoma, melanoma, multiple myeloma, mycosis fungoides, myeloid and lymphocytic leukemia, neuroblastoma, non-Hodgkin's lymphoma, osteogenic sarcoma, ovarian carcinoma, pancreatic carcinoma, polycythemia vera, primary brain carcinoma, primary macroglobulinemia, prostatic carcinoma, renal cell carcinoma, rhabdomyosarcoma, skin cancer, small-cell lung carcinoma, soft-tissue sarcoma, squamous cell carcinoma, stomach carcinoma, testicular carcinoma, thyroid carcinoma, and Wilms' tumor. In one embodiment, the cancer is a solid tumor. In another embodiment, the cancer is selected from the group consisting of colon cancer, brain cancer, glioma, multiple myeloma, head and neck cancer (except for esophageal cancer), hepatocellular cancer, melanoma, ovarian cancer, cervical cancer, renal cancer, and non-small cell lung cancer. In certain embodiments, the hyperproliferative disorder may be newly diagnosed glioblastoma multiforme or refractory anaplastic astrocytoma.

Animals which may be treated according to the present invention include all animals which may benefit from administration of compounds having Formula I. Such animals include humans, pets such as dogs and cats, and veterinary animals such as cows, pigs, sheep, goats and the like.

The term “pharmaceutical composition” as used herein, is to be understood as defining compositions of which the individual components or ingredients are themselves pharmaceutically acceptable, e.g., where oral administration is foreseen, acceptable for oral use; where topical administration is foreseen, topically acceptable; and where intravenous administration is foreseen, intravenously acceptable.

As used herein, the term “therapeutically effective amount” refers to that amount of the therapeutic agent sufficient to result in amelioration of one or more symptoms of a disorder, or prevent advancement of a disorder, or cause regression of the disorder. For example, with respect to the treatment of cancer, a therapeutically effective amount preferably refers to the amount of a therapeutic agent that decreases the rate of tumor growth, decreases tumor mass, decreases the number of metastases, increases time to tumor progression, or increases survival time by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. With respect to the treatment of metastasis, a therapeutically effective amount preferably refers to the amount of a therapeutic agent that decreases the rate of further spread of metastasis to new organs, decreases the number of metastases, increases time to metastasis progression, or increases survival time by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

The terms “prevent,” “preventing,” and “prevention,” as used herein, refer to a decrease in the occurrence of pathological cells (e.g., hyperproliferative or neoplastic cells) in an animal. The prevention may be complete, e.g., the total absence of pathological cells in a subject. The prevention may also be partial, such that the occurrence of pathological cells in a subject is less than that which would have occurred without the present invention.

As used herein, the term “metastasis” means either (1) the process by which cancer spreads from the place at which it first arose as a primary tumor to distant locations in the body, or (2) the condition produced by such spread. Metastasis depends on the cancer cells acquiring two separate abilities—increased motility and invasiveness. Invasiveness refers to the ability of the cancer to penetrate through the membranes that separate them from healthy tissues (or blood vessels) and metastasize. Metastasis is a complex multi-step process that begins with changes in the genetic material of a cell (carcinogenesis) followed by the uncontrolled multiplication of altered cells. It continues with the development of a new blood supply for the tumor (angiogenesis), invasion of the circulatory system, dispersal of small clumps of tumor cells to other organs or parts of the body, and the growth of secondary tumors in those sites. Cells that metastasize are basically of the same kind as those in the original tumor. If a cancer arises in the lung and metastasizes to the liver, the cancer cells in the liver are lung cancer cells. However, the cells have acquired increased motility and the ability to invade another organ.

It has been discovered that AQ4N reduces and sometimes prevents metastasis of implanted BxPC-3 tumors in mice. Therefore, it is expected that the compounds described herein will be useful for reducing or preventing metastasis of cancer in animals when administered alone or together with one or more other active agents or treatments. For example, the compounds described herein may be administered to an animal after it had its cancer removed surgically and/or treated with radiotherapy and/or chemotherapy.
The administration of the compounds is expected to reduce or prevent regrowth of the original cancer as well metastasis of any residual cancer cells to distant parts of the body. In another embodiment, the compounds of the invention may be administered to an animal whose cancer is considered inoperable, thus reducing or preventing the metastasis of the cancer cells.

Compounds having Formula I can be provided as pharmaceutically acceptable salts. Examples of pharmaceutically acceptable salts (i.e., addition salts) include inorganic and organic acid addition salts such as hydrochloride, hydrobromide, phosphate, sulphate, citrate, lactate, tartrate, maleate, fumarate, mandelate, benzoate and oxalate; and inorganic and organic base addition salts with bases such as sodium hydroxy, Tris(hydroxymethyl)aminomethane (TRIS, tromethane) and N-methyl-glucamine. Although the salts typically have similar physiological properties compared to the free base, certain acid addition salts may demonstrate preferred physicochemical properties, e.g., enhanced solubility, improved stability. One particular pharmaceutically acceptable salt is the maleate, such as the dimaleate.

Certain of the compounds of the present invention may exist as stereoisomers including optical isomers. The invention includes all stereoisomers and both the racemic mixtures of such stereoisomers as well as the individual enantiomers that may be separated according to methods that are well known to those of ordinary skill in the art.

In certain embodiments of the invention, compounds having Formula I are administered in combination with one or more other active agents (e.g., chemotherapeutic agents) or treatments. By way of non-limiting example, a patient may be treated for a hyperproliferative disorder, such as cancer, by the administration of a therapeutically effective amount of a compound having Formula I in combination with radiotherapy agent/treatment or the administration of a chemotherapeutic agent.

“In combination” refers to the use of more than one treatment. The use of the term “in combination” does not restrict the order in which treatments are administered to a subject being treated for a hyperproliferative disorder. A first treatment can be administered prior to, concurrently with, after, or within any cycling regimen involving the administration of a second treatment to a subject with a hyperproliferative disorder. For example, the first treatment can be administered 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before a treatment; or the first treatment can be administered 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after a second treatment. Such treatments include, for example, the administration of compounds having Formula I in combination with one or more chemotherapeutic agents or radiotherapeutic agents/treatments.

The term “chemotherapeutic agent,” as used herein, is intended to refer to any chemotherapeutic agent known to those of skill in the art to be effective for the treatment, prevention or amelioration of hyperproliferative disorders such as cancer. Chemotherapeutic agents include, but are not limited to, small molecules, synthetic drugs, peptides, polypeptides, proteins, nucleic acids (e.g., DNA and RNA polynucleotides including, but not limited to, antisense nucleotide sequences, triple helices and nucleotide sequences encoding biologically active proteins, polypeptides or peptides), antibodies, synthetic or natural inorganic molecules, mimetic agents, and synthetic or natural organic molecules. Any agent which is known to be useful, or which has been used or is currently being used for the treatment or amelioration of a hyperproliferative disorder can be used in combination with a compound having Formula I. See, e.g., Hardman et al., eds., 2002, Goodman & Gilman's The Pharmacological Basis Of Therapeutics 10th Ed, Mc-Graw-Hill, New York, N.Y. for information regarding therapeutic agents which have been or are currently being used for the treatment or amelioration of a hyperproliferative disorder.

Particular chemotherapeutic agents useful in the methods and compositions of the invention include alkylating agents, antimetabolites, anti-mitotic agents, epipodophyllotoxins, antibiotics, hormones and hormone antagonists, enzymes, platinum coordination complexes, anthracenediones, substituted ureas, methylhydrazine derivatives, imidazotetrazine derivatives, cytoprotective agents, DNA topoisomerase inhibitors, biological response modifiers, retinoids, therapeutic antibodies, differentiating agents, immunomodulatory agents, angiogenesis inhibitors and anti-angiogenic agents.

Certain chemotherapeutic agents include, but are not limited to, abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate. In certain embodiments, chemotherapeutic agents are selected from gemcitabine and irinotecan.

Chemotherapeutic agents may be administered at doses that are recognized by those of skill in the art to be effective for the treatment of the hyperproliferative disorder. In certain embodiments, chemotherapeutic agents may be administered at doses lower than those used in the art due to the additive or synergistic effect of the compounds having Formula I.

The term “radiotherapeutic agent,” as used herein, is intended to refer to any radiotherapeutic agent known to one of skill in the art to be effective to treat or ameliorate a hyperproliferative disorder, without limitation. For instance, the radiotherapeutic agent can be an agent such as those administered in brachytherapy or radionuclide therapy.

Brachytherapy can be administered according to any schedule, dose, or method known to one of skill in the art to be effective in the treatment or amelioration of a hyperproliferative disorder, without limitation. In general, brachytherapy comprises insertion of radioactive sources into the body of a subject to be treated for cancer, such as inside the tumor itself, such that the tumor is maximally exposed to the radioactive source, and minimizing the exposure of healthy tissue. Representative radioisotopes that can be administered in brachytherapy include, but are not limited to, phosphorus 32, cobalt 60, palladium 103, ruthenium 106, iodine 125, cesium 137, iridium 192, xenon 133, radium 226, californium 252, or gold 198. Methods of administering and apparatuses and compositions useful for brachytherapy are described in Mazeron et al., Sem. Rad. Onc. 12:95-108 (2002) and U.S. Pat. Nos. 6,319,189, 6,179,766, 6,168,777, 6,149,889, and 5,611,767.

Radionuclide therapy can be administered according to any schedule, dose, or method known to one of skill in the art to be effective in the treatment or amelioration of a hyperproliferative disorder, without limitation. In general, radionuclide therapy comprises systemic administration of a radioisotope that preferentially accumulates in or binds to the surface of cancerous cells. The preferential accumulation of the radionuclide can be mediated by a number of mechanisms, including, but not limited to, incorporation of the radionuclide into rapidly proliferating cells, specific accumulation of the radionuclide by the cancerous tissue without special targeting, or conjugation of the radionuclide to a biomolecule specific for a neoplasm.

Representative radioisotopes that can be administered in radionuclide therapy include, but are not limited to, phosphorus 32, yttrium 90, dysprosium 165, indium 111, strontium 89, samarium 153, rhenium 186, iodine 131, iodine 125, lutetium 177, and bismuth 213. While all of these radioisotopes may be linked to a biomolecule providing specificity of targeting, iodine 131, indium 111, phosphorus 32, samarium 153, and rhenium 186 may be administered systemically without such conjugation. One of skill in the art may select a specific biomolecule for use in targeting a particular neoplasm for radionuclide therapy based upon the cell-surface molecules present on that neoplasm. Examples of biomolecules providing specificity for particular cell are reviewed in an article by Thomas, Cancer Biother. Radiopharm. 17:71-82 (2002), which is incorporated herein by reference in its entirety. Furthermore, methods of administering and compositions useful for radionuclide therapy may be found in U.S. Pat. Nos. 6,426,400, 6,358,194, 5,766,571.

The term “radiotherapeutic treatment,” as used herein, is intended to refer to any radiotherapeutic treatment known to one of skill in the art to be effective to treat or ameliorate a hyperproliferative disorder, without limitation. For instance, the radiotherapeutic treatment can be external-beam radiation therapy, thermotherapy, radiosurgery, charged-particle radiotherapy, neutron radiotherapy, or photodynamic therapy.

External-beam radiation therapy can be administered according to any schedule, dose, or method known to one of skill in the art to be effective in the treatment or amelioration of a hyperproliferative disorder, without limitation. In general, external-beam radiation therapy comprises irradiating a defined volume within a subject with a high energy beam, thereby causing cell death within that volume. The irradiated volume preferably contains the entire cancer to be treated, and preferably contains as little healthy tissue as possible. Methods of administering and apparatuses and compositions useful for external-beam radiation therapy can be found in U.S. Pat. Nos. 6,449,336, 6,398,710, 6,393,096, 6,335,961, 6,307,914, 6,256,591, 6,245,005, 6,038,283, 6,001,054, 5,802,136, 5,596,619, and 5,528,652.

Fractionated radiotherapy can be administered according to any schedule, dose, or method known to one of skill in the art to be effective in the treatment or amelioration of a hyperproliferative disorder, without limitation. In general, external-beam radiation delivers a fraction of the complete radiation dose over many sessions to shrink or destroy tumors. Delivering a fraction of the radiation dose over many sessions will allow normal cells time to repair themselves between treatments and are protected from permanent injury or death.

Thermotherapy can be administered according to any schedule, dose, or method known to one of skill in the art to be effective in the treatment or amelioration of a hyperproliferative disorder, without limitation. In certain embodiments, the thermotherapy can be cryoablation therapy. In other embodiments, the thermotherapy can be hyperthermic therapy. In still other embodiments, the thermotherapy can be a therapy that elevates the temperature of the tumor higher than in hyperthermic therapy.

Cryoablation therapy involves freezing of a neoplastic mass, leading to deposition of intra- and extra-cellular ice crystals; disruption of cellular membranes, proteins, and organelles; and induction of a hyperosmotic environment, thereby causing cell death. Methods for and apparatuses useful in cryoablation therapy are described in Murphy et al., Sem. Urol. Oncol. 19:133-140 (2001) and U.S. Pat. Nos. 6,383,181, 6,383,180, 5,993,444, 5,654,279, 5,437,673, and 5,147,355.

Hyperthermic therapy typically involves elevating the temperature of a neoplastic mass to a range from about 42° C. to about 44° C. The temperature of the cancer may be further elevated above this range; however, such temperatures can increase injury to surrounding healthy tissue while not causing increased cell death within the tumor to be treated. The tumor may be heated in hyperthermic therapy by any means known to one of skill in the art without limitation. For example, and not by way of limitation, the tumor may be heated by microwaves, high intensity focused ultrasound, ferromagnetic thermoseeds, localized current fields, infrared radiation, wet or dry radiofrequency ablation, laser photocoagulation, laser interstitial thermic therapy, and electrocautery. Microwaves and radiowaves can be generated by waveguide applicators, horn, spiral, current sheet, and compact applicators.

Other methods, apparatuses and compositions for raising the temperature of a tumor are reviewed in an article by Wust et al., Lancet Oncol. 3:487-97 (2002), and described in U.S. Pat. Nos. 6,470,217, 6,379,347, 6,165,440, 6,163,726, 6,099,554, 6,009,351, 5,776,175, 5,707,401, 5,658,234, 5,620,479, 5,549,639, and 5,523,058.

Radiosurgery can be administered according to any schedule, dose, or method known to one of skill in the art to be effective in the treatment or amelioration of a hyperproliferative disorder, without limitation. In general, radiosurgery comprises exposing a defined volume within a subject to a manually directed radioactive source, thereby causing cell death within that volume. The irradiated volume preferably contains the entire cancer to be treated, and preferably contains as little healthy tissue as possible. Typically, the tissue to be treated is first exposed using conventional surgical techniques, then the radioactive source is manually directed to that area by a surgeon. Alternatively, the radioactive source can be placed near the tissue to be irradiated using, for example, a laparoscope. Methods and apparatuses useful for radiosurgery are further described in Valentini et al., Eur. J. Surg. Oncol. 28:180-185 (2002) and in U.S. Pat. Nos. 6,421,416, 6,248,056, and 5,547,454.

Charged-particle radiotherapy can be administered according to any schedule, dose, or method known to one of skill in the art to be effective in the treatment or amelioration of a hyperproliferative disorder, without limitation. In certain embodiments, the charged-particle radiotherapy can be proton beam radiotherapy. In other embodiments, the charged-particle radiotherapy can be helium ion radiotherapy. In general, charged-particle radiotherapy comprises irradiating a defined volume within a subject with a charged-particle beam, thereby causing cellular death within that volume. The irradiated volume preferably contains the entire cancer to be treated, and preferably contains as little healthy tissue as possible. A method for administering charged-particle radiotherapy is described in U.S. Pat. No. 5,668,371.

Neutron radiotherapy can be administered according to any schedule, dose, or method known to one of skill in the art to be effective in the treatment or amelioration of a hyperproliferative disorder, without limitation. In certain embodiments, the neutron radiotherapy can be a neutron capture therapy. In such embodiments, a compound that emits radiation when bombarded with neutrons and preferentially accumulates in a neoplastic mass is administered to a subject. Subsequently, the tumor is irradiated with a low energy neutron beam, activating the compound and causing it to emit decay products that kill the cancerous cells. The compound to be activated can be caused to preferentially accumulate in the target tissue according to any of the methods useful for targeting of radionuclides, as described above, or in the methods described in Laramore, Semin. Oncol. 24:672-685 (1997) and in U.S. Pat. Nos. 6,400,796, 5,877,165, 5,872,107, and 5,653,957.

In other embodiments, the neutron radiotherapy can be a fast neutron radiotherapy. In general, fast neutron radiotherapy comprises irradiating a defined volume within a subject with a neutron beam, thereby causing cellular death within that volume.

Photodynamic therapy can be administered according to any schedule, dose, or method known to one of skill in the art to be effective in the treatment or amelioration of cancer, without limitation. In general, photodynamic therapy comprises administering a photosensitizing agent that preferentially accumulates in a neoplastic mass and sensitizes the neoplasm to light, then exposing the tumor to light of an appropriate wavelength. Upon such exposure, the photosensitizing agent catalyzes the production of a cytotoxic agent, such as, e.g., singlet oxygen, which kills the cancerous cells. Methods of administering and apparatuses and compositions useful for photodynamic therapy are disclosed in Hopper, Lancet Oncol. 1:212-219 (2000) and U.S. Pat. Nos. 6,283,957, 6,071,908, 6,011,563, 5,855,595, 5,716,595, and 5,707,401.

Radiotherapy can be administered to destroy hyperproliferative cells before or after surgery, before or after chemotherapy, and sometimes during chemotherapy. Radiotherapy may also be administered for palliative reasons to relieve symptoms of a hyperproliferative disorder, for example, to lessen pain. Among the types of tumors that can be treated using radiotherapy are localized tumors that cannot be excised completely and metastases and tumors whose complete excision would cause unacceptable functional or cosmetic defects or be associated with unacceptable surgical risks.

It will be appreciated that both the particular radiation dose to be utilized in treating a hyperproliferative disorder and the method of administration will depend on a variety of factors. Thus, the dosages of radiation that can be used according to the methods of the present invention are determined by the particular requirements of each situation. The dosage will depend on such factors as the size of the tumor, the location of the tumor, the age and sex of the patient, the frequency of the dosage, the presence of other tumors, possible metastases and the like. Those skilled in the art of radiotherapy can readily ascertain the dosage and the method of administration for any particular tumor by reference to Hall, E. J., Radiobiology for the Radiologist, 5th edition, Lippincott Williams & Wilkins Publishers, Philadelphia, Pa., 2000; Gunderson, L. L. and Tepper J. E., eds., Clinical Radiation Oncology, Churchill Livingstone, London, England, 2000; and Grosch, D. S., Biological Effects of Radiation, 2nd edition, Academic Press, San Francisco, Calif., 1980. In certain embodiments, radiotherapeutic agents and treatments may be administered at doses lower than those known in the art due to the additive or synergistic effect of the compound having Formula I.

Compositions in accordance with the present invention may be employed for administration in any appropriate manner, e.g., oral or buccal administration, e.g., in unit dosage form, for example in the form of a tablet, in a solution, in hard or soft encapsulated form including gelatin encapsulated form, sachet, or lozenge. Compositions may also be administered parenterally or topically, e.g., for application to the skin, for example in the form of a cream, paste, lotion, gel, ointment, poultice, cataplasm, plaster, dermal patch or the like, or for ophthalmic application, for example in the form of an eye-drop, -lotion or -gel formulation. Readily flowable forms, for example solutions, emulsions and suspensions, may also be employed e.g., for intralesional injection, or may be administered rectally, e.g., as an enema or suppository, or intranasal administration, e.g., as a nasal spray or aerosol. Microcrystalline powders may be formulated for inhalation, e.g., delivery to the nose, sinus, throat or lungs. Transdermal compositions/devices and pessaries may also be employed for delivery of the compounds of the invention. The compositions may additionally contain agents that enhance the delivery of the compounds having Formula I (or other active agents), e.g., liposomes, polymers or co-polymers (e.g., branched chain polymers). Preferred dosage forms of the present invention include oral dosage forms and intravenous dosage forms.

Intravenous forms include, but are not limited to, bolus and drip injections. In preferred embodiments, the intravenous dosage forms are sterile or capable of being sterilized prior to administration to a subject since they typically bypass the subject's natural defenses against contaminants. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

The pharmaceutical compositions of the present invention may further comprise one or more additives. Additives that are well known in the art include, e.g., detackifiers, anti-foaming agents, buffering agents, antioxidants (e.g., ascorbic acid, ascorbyl palmitate, sodium ascorbate, butylated hydroxyanisole (BHA), butylated hydroxytoluene (BHT), propyl gallate, malic acid, fumaric acid, potassium metabisulfite, sodium bisulfite, sodium metabisulfite, and tocopherols, e.g., α-tocopherol (vitamin E)), preservatives, chelating agents, viscomodulators, tonicifiers, flavorants, colorants, odorants, opacifiers, suspending agents, binders, fillers, plasticizers, lubricants, and mixtures thereof. The amounts of such additives can be readily determined by one skilled in the art, according to the particular properties desired, and can be formulated such that compounds having Formula I are stable, e.g., not reduced by antioxidant additives.

The additive may also comprise a thickening agent. Suitable thickening agents may be of those known and employed in the art, including, e.g., pharmaceutically acceptable polymeric materials and inorganic thickening agents. Exemplary thickening agents for use in the present pharmaceutical compositions include polyacrylate and polyacrylate co-polymer resins, for example poly-acrylic acid and poly-acrylic acid/methacrylic acid resins; celluloses and cellulose derivatives including: alkyl celluloses, e.g., methyl-, ethyl- and propyl-celluloses; hydroxyalkyl-celluloses, e.g., hydroxypropyl-celluloses and hydroxypropylalkyl-celluloses such as hydroxypropyl-methyl-celluloses; acylated celluloses, e.g., cellulose-acetates, cellulose-acetatephthallates, cellulose-acetatesuccinates and hydroxypropylmethyl-cellulose phthallates; and salts thereof such as sodium-carboxymethyl-celluloses; polyvinylpyrrolidones, including for example poly-N-vinylpyrrolidones and vinylpyrrolidone co-polymers such as vinylpyrrolidone-vinylacetate co-polymers; polyvinyl resins, e.g., including polyvinylacetates and alcohols, as well as other polymeric materials including gum traganth, gum arabicum, alginates, e.g., alginic acid, and salts thereof, e.g., sodium alginates; and inorganic thickening agents such as atapulgite, bentonite and silicates including hydrophilic silicon dioxide products, e.g., alkylated (for example methylated) silica gels, in particular colloidal silicon dioxide products.

Such thickening agents as described above may be included, e.g., to provide a sustained release effect. However, where oral administration is intended, the use of thickening agents may not be required. Use of thickening agents is, on the other hand, indicated, e.g., where topical application is foreseen.

In one embodiment of the invention, compounds having Formula I are formulated as described in WO 03/076387. In particular, the compounds are formulated such that upon dissolution in aqueous solution the pH of the solution is in the range of 5 to 9.

Although the dosage of the compound having Formula I will vary according to the activity and/or toxicity of the particular compound, the condition being treated, and the physical form of the pharmaceutical composition being employed for administration, it may be stated by way of guidance that a dosage selected in the range from 0.1 to 20 mg/kg of body weight per day will often be suitable, although higher dosages, such as 0.1 to 50 mg/kg of body weight per day may be useful. Those of ordinary skill in the art are familiar with methods for determining the appropriate dosage. Methods for assessing the toxicity, activity and/or selectivity of the compounds having Formula I may be carried out as described in Lee et al., supra, and PCT Published International Application WO 92/15300, supra, and may be useful for approximating and/or determining dose ranges for compounds having Formula I.

In certain instances, the dosage of the compounds having Formula I will be lower, e.g., when used in combination with at least a second hyperproliferative disorder treatment, and may vary according to the activity and/or toxicity of the particular compound, the condition being treated, and the physical form of the pharmaceutical composition being employed for administration.

When the composition of the present invention is formulated in unit dosage form, the compound having Formula I will preferably be present in an amount of between 0.01 and 2000 mg per unit dose. More preferably, the amount of compound having Formula I per unit dose will be about 0.01, 0.05, 0.1, 0.5, 1, 5, 10, 20, 30, 40, 50, 60, 70, 80, 90, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or 2000 mg or any amount therein.

When the unit dosage form of the composition is a capsule, the total quantity of ingredients present in the capsule is preferably about 10-1000 μL. More preferably, the total quantity of ingredients present in the capsule is about 100-300 μL. In another embodiment, the total quantity of ingredients present in the capsule is preferably about 10-1500 mg, preferably about 100-1000 mg.

The relative proportion of ingredients in the compositions of the invention will, of course, vary considerably depending on the particular type of composition concerned. The relative proportions will also vary depending on the particular function of ingredients in the composition. The relative proportions will also vary depending on the particular ingredients employed and the desired physical characteristics of the product composition, e.g., in the case of a composition for topical use, whether this is to be a free flowing liquid or a paste. Determination of workable proportions in any particular instance will generally be within the capability of a person of ordinary skill in the art. All indicated proportions and relative weight ranges described below are accordingly to be understood as being indicative individually inventive teachings only and not as not limiting the invention in its broadest aspect.

The amount of compound having Formula I in compositions of the invention will of course vary, e.g., depending on the intended route of administration and to what extent other components are present. In general, however, the compound having Formula I will suitably be present in an amount of from about 0.005% to 20% by weight based upon the total weight of the composition. In certain embodiments, the compound having Formula I is present in an amount of from about 0.01% to 15% by weight based upon the total weight of the composition.

In addition to the foregoing, the present invention also provides a process for the production of a pharmaceutical composition as hereinbefore defined, which process comprises bringing the individual components thereof into intimate admixture and, when required, compounding the obtained composition in unit dosage form, for example filling said composition into tablets, gelatin, e.g., soft or hard gelatin, capsules, or non-gelatin capsules.

Compounds having Formula I can be prepared by methods well known in the art and as disclosed in U.S. Pat. No. 5,132,327.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### EXAMPLE 1

### Cytotoxicity of AQ4 and AQ4N in Lymphoma, Leukemia, and Multiple Myeloma

The cytotoxicity of AQ4 and AQ4N on different lymphoma, leukemia, and multiple myeloma cell lines was tested in vitro under normoxic conditions. Standard cytotoxicity assays using MTS dye were run to determine the IC₅₀ for each compound. Cells were exposed to the compounds for 24 hours and cells were stained 24-72 hours post-drug exposure. Positive controls utilized chemotherapeutic agents at doses shown in the art to be effective. As shown in Table 1, the results indicate that AQ4 is cytotoxic to many of the cell lines, with IC₅₀ values in the nanomolar to sub-nanomolar range. AQ4N was less active or inactive compared to AQ4, but the tests were done under normoxic conditions, so it is expected that there is little conversion of AQ4N to AQ4 under these conditions. In most instances, AQ4 was at least as cytotoxic as the standard chemotherapeutic agent.

TABLE 1
Cell cytotoxicity in lymphoma, leukemia, and multiple myeloma

| | | AQ4 | AQ4N | |
|---|---|---|---|---|
| Tumor Line | Type | (IC₅₀) | (IC₅₀) | Standard |
| Daudi | Burkitt | 4.6 nM | NA | 0.5 nM |
| | Lymphoma | | | Dox |
| Raji | Burkitt | 200 nM | NA | 0.9 nM |
| | Lymphoma | | | Dox |
| Ramos | Burkitt | 8.0 nM | NA | 1.8 nM |
| | Lymphoma | | | Dox |
| Namalwa | Burkitt | 0.2 nM | 400 nM | 7.4 nM |
| | Lymphoma | | | Dox |
| MOLT-4 | ALL | 2 nM | 700 nM | 7.5 nM |
| | (human) | | | Dox |
| HL-60 | AML | 10 nM | NA | 100 nM |
| | (human) | | | Dox |
| KG1a | AML | 50 nM | 43 μM | 600 nM |
| | (human) | | | Dox |
| K562 | CML | 400 nM | 1000 nM | 200 nM |
| | (human) | | | Dox |
| P388 | CLL | 10 nM | 31.5 μM | 100 nM |
| | (mouse) | | | Dox |
| L1210 | ALL | 1.2 nM | 600 nM | 50 nM |
| | (mouse) | | | Dox |
| CCRF-CEM | T-ALL | 620 nM | 237.5 μM | 10 nM |
| | | | | VLB |
| CCRF-CEM/VLB | T-ALL | 340 nM | 310 μM | 1041 μM |
| | | | | VLB |
| L5178Y | Mouse | 30 nM | 300 nM | 50 nM |
| | Lymphoma | | | Dox |
| RPMI8226 | Multiple | 200 nM | NA | 100 nM |
| | Myeloma | | | Dox |
| RPMI8226/Dox | Multiple | 1100 nM | NA | 54.7 μM |
| | Myeloma | | | Dox |
| ARH177 | Multiple | 200 nM | NA | — |
| | Myeloma | | | |

ALL—acute lymphocytic leukemia;
AML—acute myelogenous leukemia;
CML—chronic myelogenous leukemia;
CLL—chronic lymphocytic leukemia;
T-ALL—T cell acute lymphocytic leukemia;
Dox—doxorubicin;
VLB—vinorelbine;
NA—not active (IC₅₀ >100 μM)

### EXAMPLE 2

### Cytotoxicity of AQ4N in Lymphoma and Multiple Myeloma In Vivo

The cytotoxic effects of AQ4N on lymphoma and multiple myeloma were tested in vivo using a tumor model. Tumor cells were implanted intraperitoneally in mice and various treatment schedules for AQ4N were tested. Animals were monitored for survival time. Standard doses of other chemotherapeutic agents were used as controls.

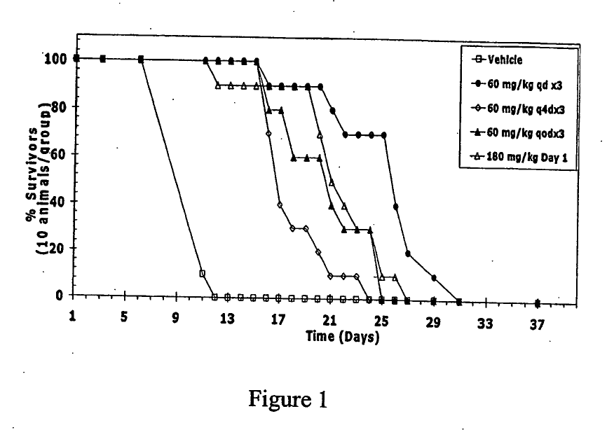

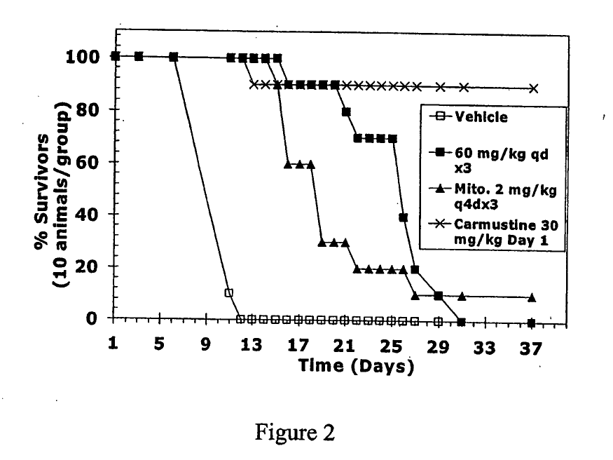

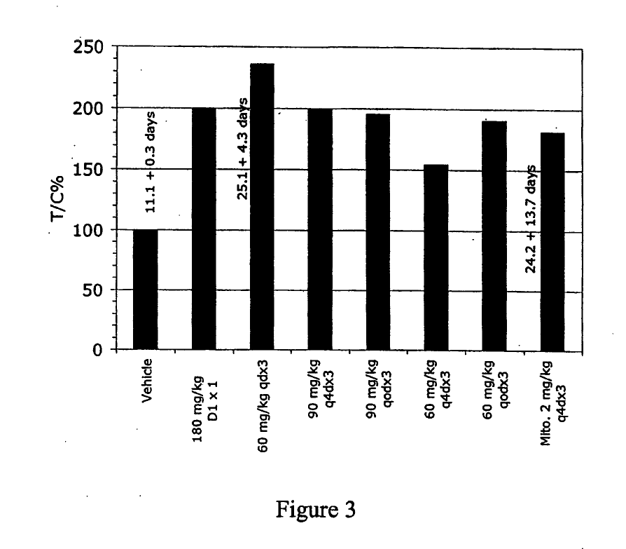

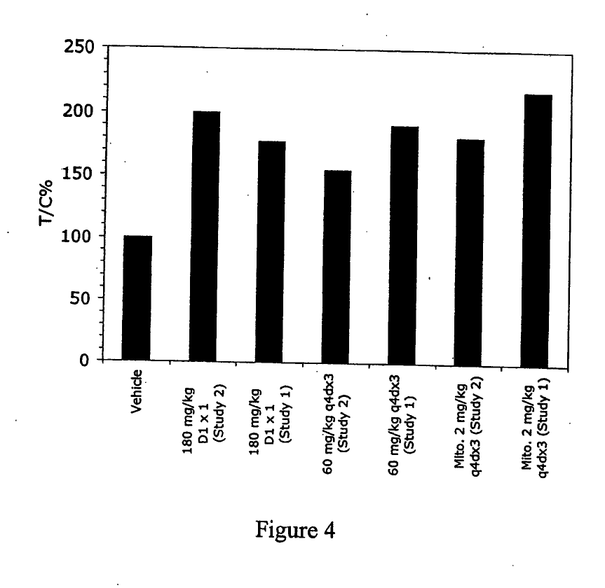

Using a P388 murine CLL model, the administration of AQ4N was shown to increase survival time (FIG. 1). Survival was shown to correlate with increased initial expose to AQ4N, as administration of 60 mg/kg qd×3 promoted survival to a greater extent than administration of 180 mg/kg on Day 1 or 60 mg/kg qod×3, which in turn were more effective than 60 mg/kg q4d×4 (FIG. 1). AQ4N was also shown to be more effective in promoting survival than mitoxantrone (FIG. 2). When the data is analyzed in terms of survival time, AQ4N was shown to be at least as effective as mitoxantrone (FIG. 3) and to provide reproducible results (FIG. 4).

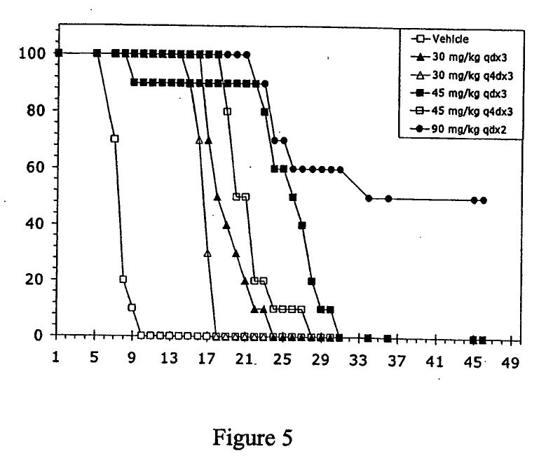

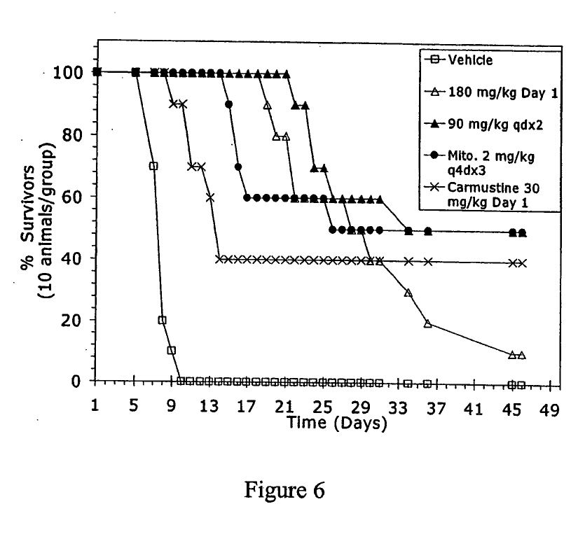

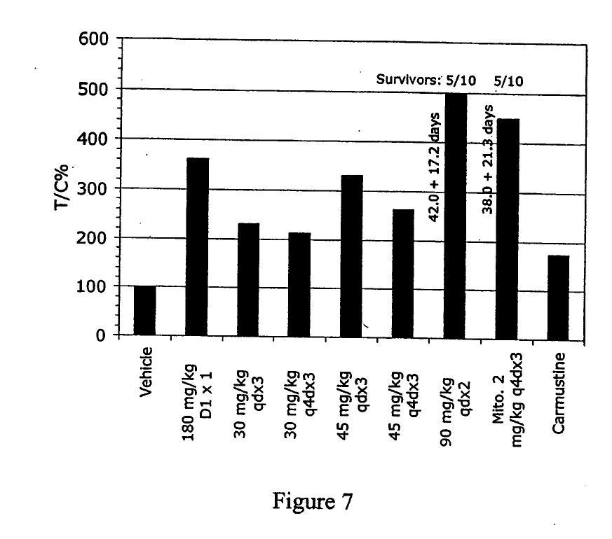

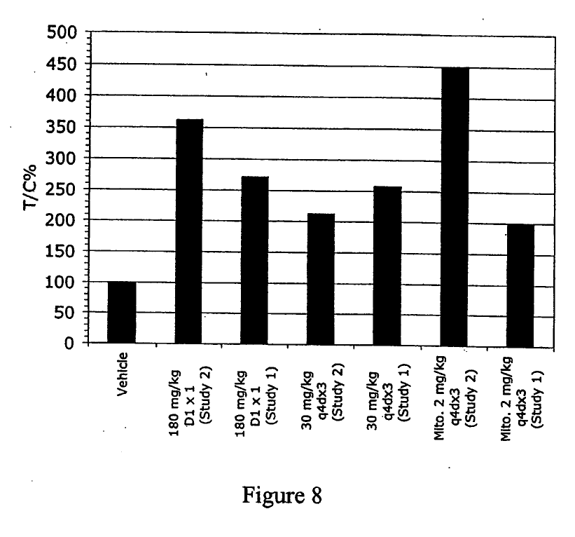

Using a L1210 murine ALL model, the administration of AQ4N was shown to increase survival time (FIG. 5). Again, survival was shown to correlate with increased initial expose to AQ4N, as administration of 90 mg/kg qd×2 promoted survival to a greater extent than administration of 45 mg/kg qd×3, which in turn were more effective than 45 mg/kg q4d×3 or 30 mg/kg on either schedule (FIG. 5). AQ4N at 90 mg/kg qd×2 was also shown to be about as effective in promoting survival as mitoxantrone or carmustine (FIG. 6). When the data is analyzed in terms of survival time, AQ4N was shown to be at least as effective as mitoxantrone and more effective than carmustine (FIG. 7) and to provide reproducible results (FIG. 8).

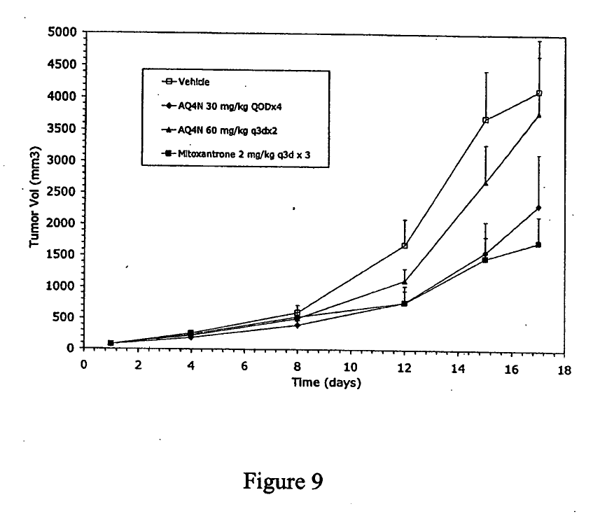

Using a Namalwa human lymphoma model, the administration of AQ4N was shown to inhibit tumor growth (FIG. 9). AQ4N at 60 mg/kg q3d×2 was also shown to be about as effective in inhibiting tumor growth as mitoxantrone (FIG. 9).

### EXAMPLE 3

### Cytotoxicity of AQ4 and AQ4N in Solid Tumors

The cytotoxicity of AQ4 and AQ4N on different solid tumor cell lines was tested in vitro under normoxic conditions. Standard cytotoxicity assays using MTS dye were run to determine the IC₅₀ for each compound. Cells were exposed to the compounds for 24 hours and cells were stained 24-72 hours post-drug exposure. Positive controls utilized chemotherapeutic agents at doses shown in the art to be effective. As shown in Table 2, the results indicate that AQ4 is cytotoxic to many of the cell lines, with IC₅₀ values in the nanomolar to sub-nanomolar range. AQ4N was less active or inactive compared to AQ4, but the tests were done under normoxic conditions, so it is expected that there is little conversion of AQ4N to AQ4 under these conditions. In many instances, AQ4 was at least as cytotoxic as the standard chemotherapeutic agent.

TABLE 2
Cell cytotoxicity in solid tumors

| | | AQ4 | AQ4N | |
|---|---|---|---|---|
| Tumor Line | Type | (IC₅₀) | (IC₅₀) | Standard |
| BXPC-3 | Pancreatic | 1.6 μM | 3.6 μM | 59.5 nM |
| | | | | Gem |
| MiaPaCa | Pancreatic | 1.6 μM | NA | 23 nM |
| | | | | Gem |
| Panc-1 | Pancreatic | 0.4 μM | NA | 1.7 μM |
| | | | | Gem |
| HT-29 | Colon | 0.7 μM | 101.5 μM | 65.1 nM |
| | | | | SN38 |
| HCT116 | Colon | 3.9 μM | NA | 48.4 nM |
| | | | | SN38 |
| LoVo | Colon | 0.21 μM | 49.9 μM | 0.6 nM |
| | | | | SN38 |
| LS174T | Colon | 0.95 μM | 14.4 μM | 2.6 nM |
| | | | | SN38 |
| U87MG | Glioma | 0.9 μM | NA | 0.4 μM |
| | | | | Dox |
| U118MG | Glioma | 1.3 μM | NA | 0.03 μM |
| | | | | Dox |
| U251 | Glioma | 0.6 μM | NA | 0.03 μM |
| | | | | Dox |
| FaDu | Pharynx Squamous | 0.3 μM | 64.7 μM | 6.5 μM |
| | Cell Carcinoma | | | Taxol |
| KB | Mouth Esophageal | 0.6 μM | 13.4 μM | 2.0 μM |
| | | | | Taxol |
| KB-3 | Mouth Esophageal | 3.3 μM | NA | 19.1 μM |
| | (radiation-resistant) | | | Taxol |
| Hep3B2.1-7 | Hepatocellular | 0.8 μM | NA | 13.7 μM |
| | | | | Taxol |
| A375-SM | Melanoma | 3.30 μM | NA | None |
| | (mouse) | | | |
| B16-F10 | Melanoma | 0.02 μM | NA | None |
| | (human) | | | |

Gem—gemcitabine;
SN38—7-ethyl-10-hydro-camptothecin;
Dox—doxorubicin;
NA—not active (IC₅₀ >100 μM)

### EXAMPLE 4

### Cytotoxicity of AQ4N in Solid Tumors In Vivo

The cytotoxic effects of AQ4N on solid tumors were tested in vivo using a mouse tumor model. Tumor cells were implanted subcutaneously in mice and allowed to grow until about 50-100 mm³ in size (10-17 days). Various treatment schedules for AQ4N were tested and animals were monitored for tumor volume. Standard doses of other chemotherapeutic agents were used as controls.

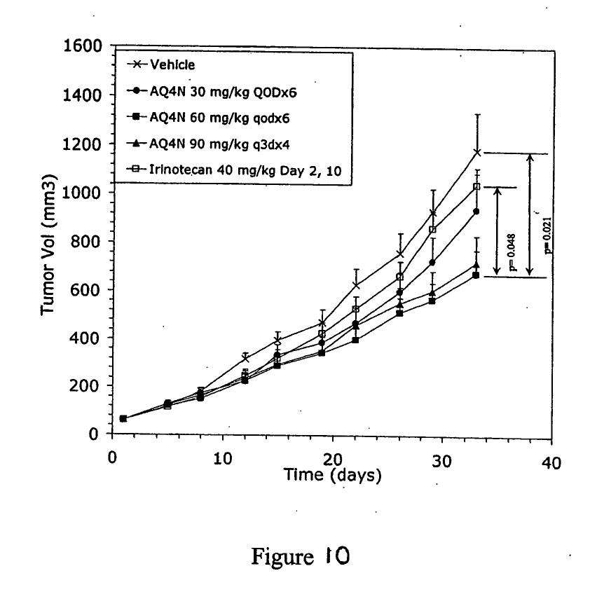

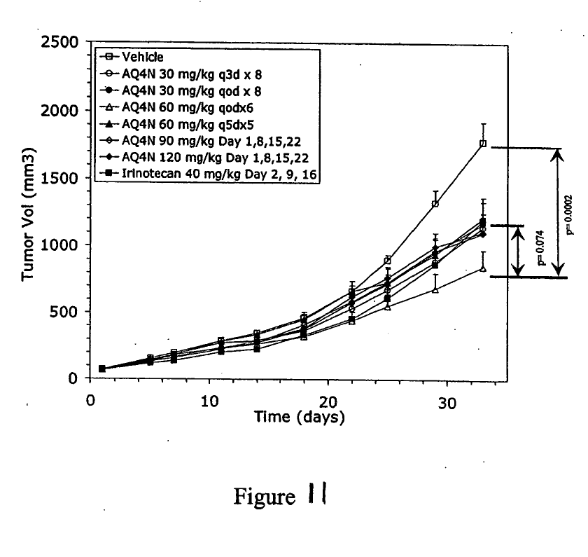

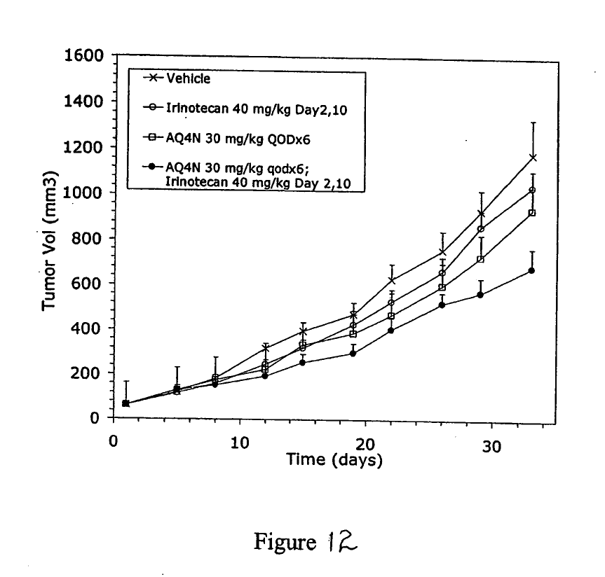

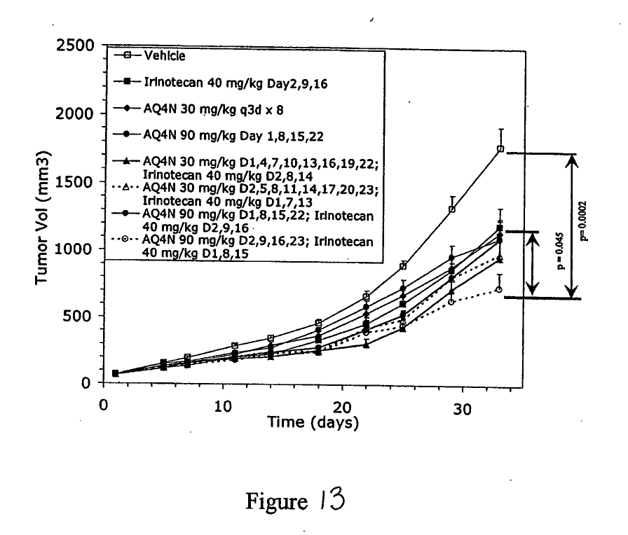

Using a HT-29 colon cancer model, the administration of increasing doses of AQ4N was shown to inhibit tumor growth, with 60 mg/kg qod×6 having a significant (p=0.021) effect on tumor growth inhibition compared to untreated controls and having significantly (p=0.048) more tumor growth inhibition than two doses of irinotecan (FIG. 10). In a further refinement of dosing schedules, administration of AQ4N as 60 mg/kg qod×6 was not significantly (p=0.074) more potent than three doses of irinotecan (FIG. 11) When AQ4N and irinotecan administration was combined, the combination caused greater tumor growth inhibition than administration of AQ4N or irinotecan alone (FIG. 12). Further testing of the combination treatment showed that a combination of AQ4N 90 mg/kg on days 2, 8, 16, and 23 with irinotecan 40 mg/kg on days 1, 8, and 15 provided the most effective results with significant (p=0.045) tumor growth inhibition compared to either agent alone (FIG. 13).

### EXAMPLE 5

### Tissue and Tumor Specificity of AQ4N

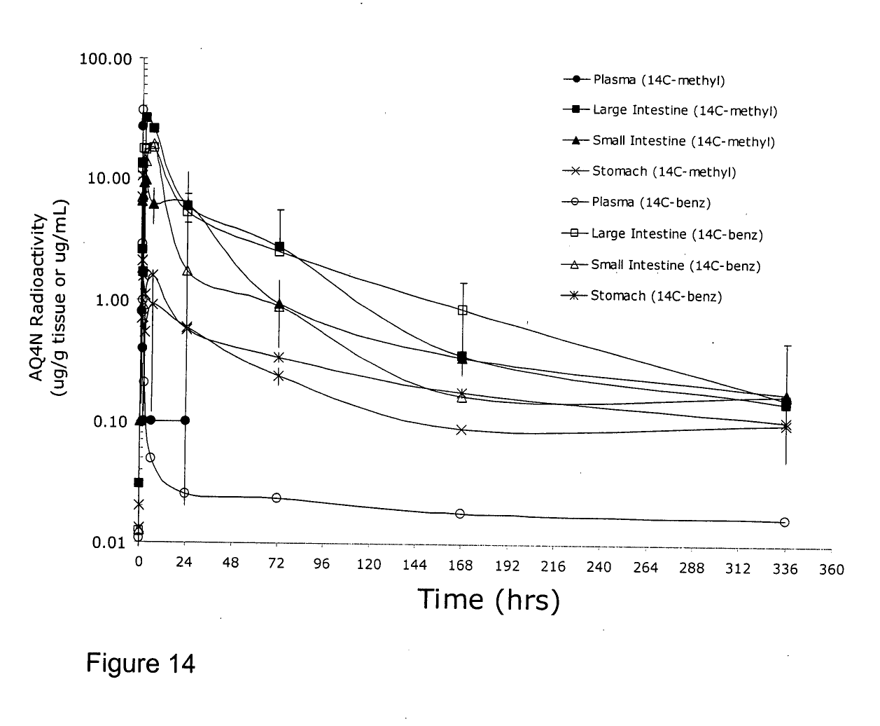

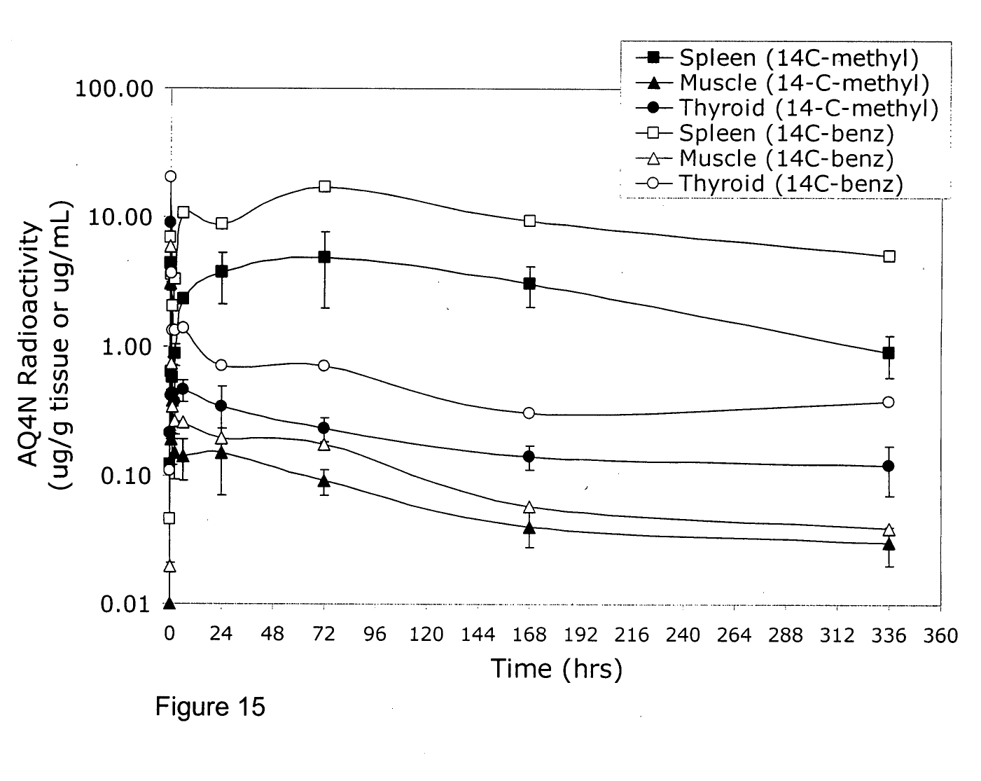

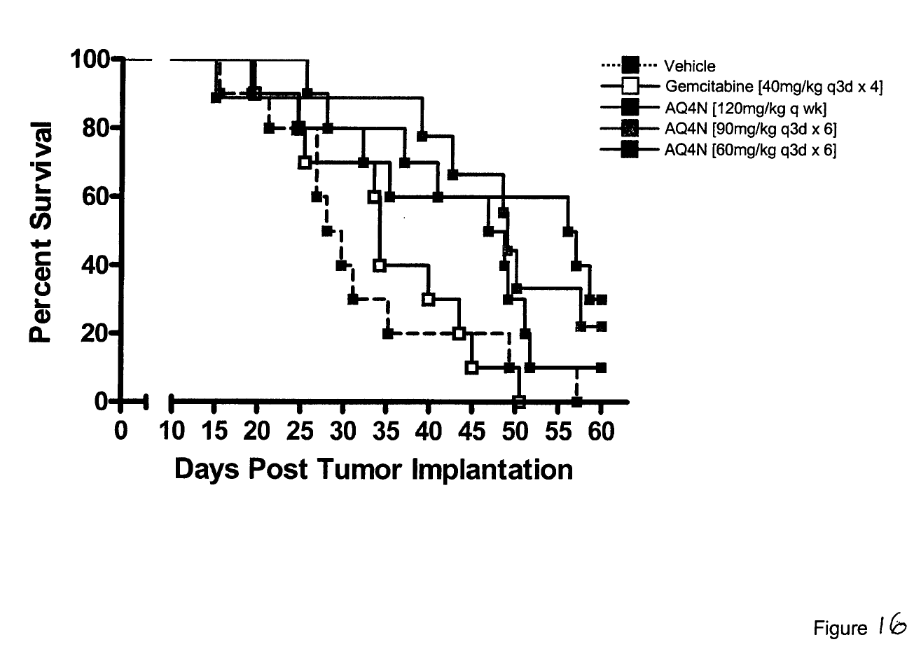

In order to determine the distribution of AQ4N after administration to a subject, AQ4N labeled with ¹⁴C on both a benzene ring and a methyl group was administered to a mouse having a subcutaneous BXPC-3 pancreatic cancer tumor (20 mg/kg; 120 μCi/kg) and the distribution of radioactivity monitored. The results (shown in Table 3) indicate that AQ4N radioactivity accumulates disproportionately in the liver, spleen, large intestine, kidney, and pancreas. The time course of radioactivity distribution indicates the accumulation of AQ4N in the large intestine, suggesting enhanced usefulness for the treatment of colon cancer (FIG. 14). The long half-life of radiolabeled AQ4N, particularly in the spleen, suggests that AQ4N may be effective even with less frequent dosing (FIG. 15).

TABLE 3
Distribution of labeled AQ4N
Exposure (μg/mL (or g) · hr)

| Tissue | ¹⁴C-Benzene | ¹⁴C-Methyl |
|---|---|---|
| Plasma | 14.1 | 14.0 |
| Subcutaneous Tumor | 142.0 | 79.0 |
| Spleen | 1592.0 | 986.0 |
| Large Intestine | 652.8 | 813.0 |
| Liver | 2089.0 | 530.0 |
| Kidney | 632.5 | 249.0 |
| Brain | 82.8 | 68.0 |
| Pancreas | 268.9 | 133.0 |

### EXAMPLE 6

### Anti-Tumor Efficacy of AQ4N in Multiple Pancreatic Xenograft Models

Based on the potent growth inhibitory activity of AQ4 demonstrated in the pancreatic tumor cell lines in vitro, we explored the anti-tumor activity of AQ4N in vivo using human Panc-1 or BxPC-3 pancreatic xenograft models. In each case, a range of AQ4N dose schedules were evaluated to determine the optimum dosage for efficacy as a single agent. Gemcitabine, a pyrimidine antimetabolite currently used to treat advanced and metastatic pancreatic cancers, was used as a comparison for AQ4N activity in these models.

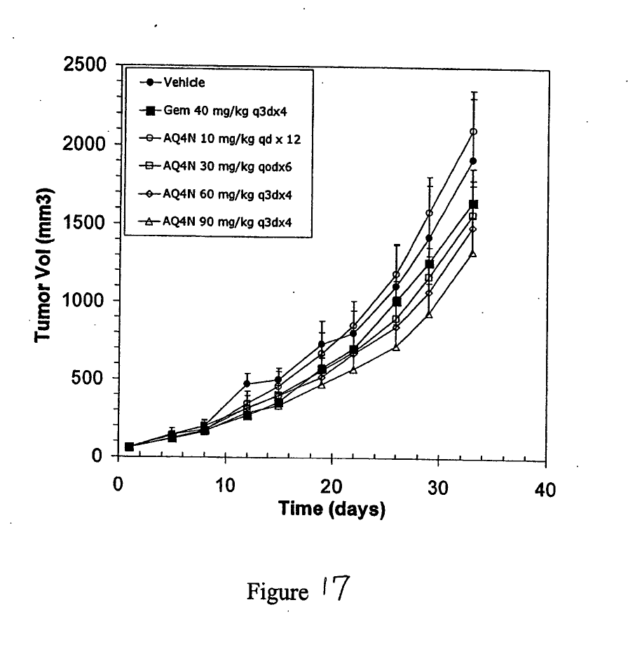

In the Panc-1 model, the endpoint of the study was pre-defined to be when tumors reached 1200 mm³ and treatment outcome was determined from tumor growth delay (TGD). TGD is defined as the increase in median time to endpoint (TTE) in a treatment group as compared to the control group, and expressed in days or as a percentage of the median TTE vs. the control group. As shown in FIG. 16, AQ4N was observed to significantly inhibit the growth of Panc-1 tumors in vivo. AQ4N-dosed mice at 90 mg/kg q3d×6 (administered once every 3 days for a total of 6 times) or 120 mg/kg weekly×8 resulted in a TGD of 20.2 day (70%; P=0.02) and 27.7 day (96%; P=0.02), respectively, compared to the vehicle control group. Furthermore, mice treated with AQ4N demonstrated a significant increase in the median time to endpoint (TTE) (49 days for AQ4N 90 mg/kg q3d×6; 56.5 days for AQ4N 120 mg/kg weekly×8) compared to the vehicle control-treated animals (28.8 days) (FIG. 16). In addition, both AQ4N treatment groups had significantly greater (P=0.02) anti-tumor activity compared to the standard agent, gemcitabine, in this model. Mice treated with gemcitabine had a TTE of 34.2 days resulting in a non-significant TGD of 5.4 days (19%) (FIG. 16).

Treatment of mice with AQ4N at 90 mg/kg q3d×6 was well-tolerated as evident by a maximum body weight loss of only 4.3% observed on day 2. This was similar to gemcitabine treatment which produced a maximum body weight loss of 5.3% on day 3. The AQ4N 120 mg/kg weekly×8 schedule was tolerated for the initial 6 treatments as evident by minimal body weight loss, however, the last two treatments were less tolerated and resulted in 17.3% weight loss at day 52. Therefore, a shorter-term schedule of weekly AQ4N dosing was used in subsequent studies.

To extend our observations in other pancreatic tumor types and to explore the effects of AQ4N on metastatis, we then studied the human BxPC-3 pancreatic carcinoma model. Initially, we examined the effects of AQ4N or gemcitabine on pre-established (60-80 mm³) BxPC-3 tumors grown subcutaneously using tumor growth inhibition (TGI) as the readout. The percent TGI, defined as the change in mean tumor weight of the treated groups/the change in mean tumor weight of the control group×100 (ΔT/ΔC), was calculated for each group when tumors from the control group reached an average of 2 g (˜day 27).

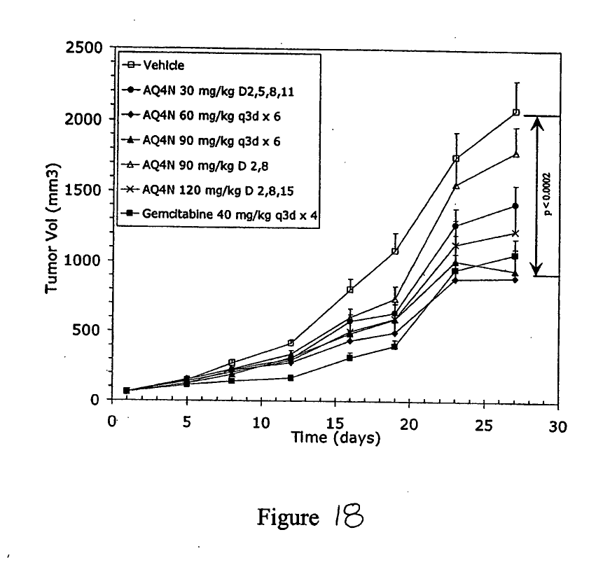

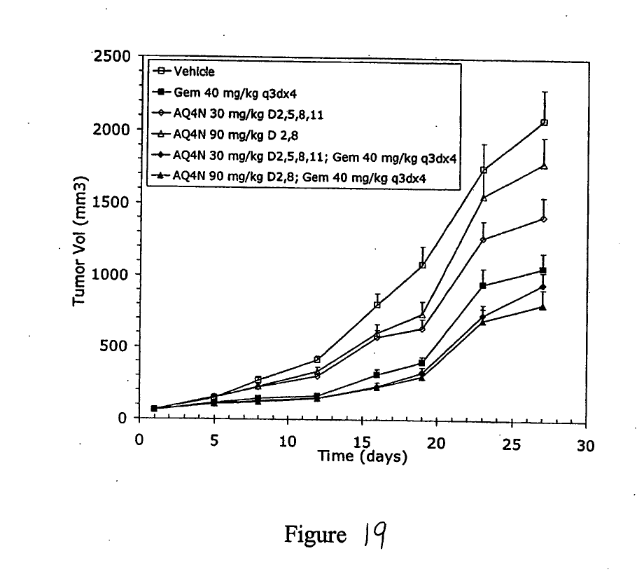

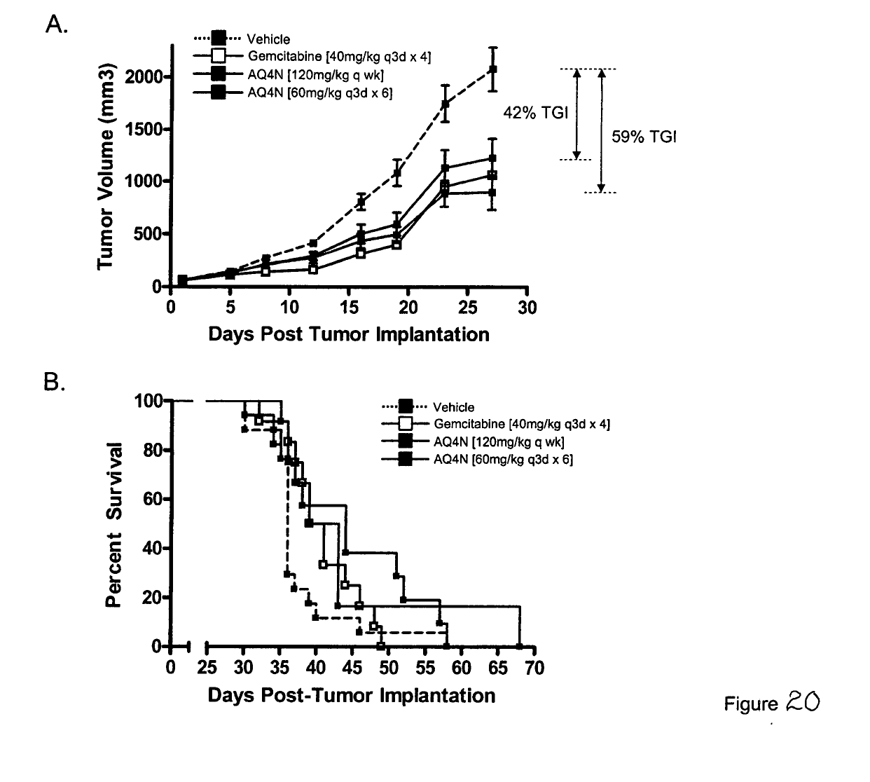

Using a BXPC-3 pancreatic cancer model, the administration of increasing doses of AQ4N was shown to inhibit tumor growth, with 90 mg/kg q3d×4 being approximately as effective as gemcitabine (FIG. 17). In a further refinement of dosing schedules, administration of AQ4N as 60 mg/kg q3d×6 and 90 mg/kg q3d×6 was shown to provide enhanced results that were statistically significant (p<0.0002) compared to the untreated control and had potency comparable to gemcitabine (FIG. 18). When AQ4N and gemcitabine administration was combined, the combination was shown to be slightly better than administration of AQ4N or gemcitabine alone (FIG. 19).

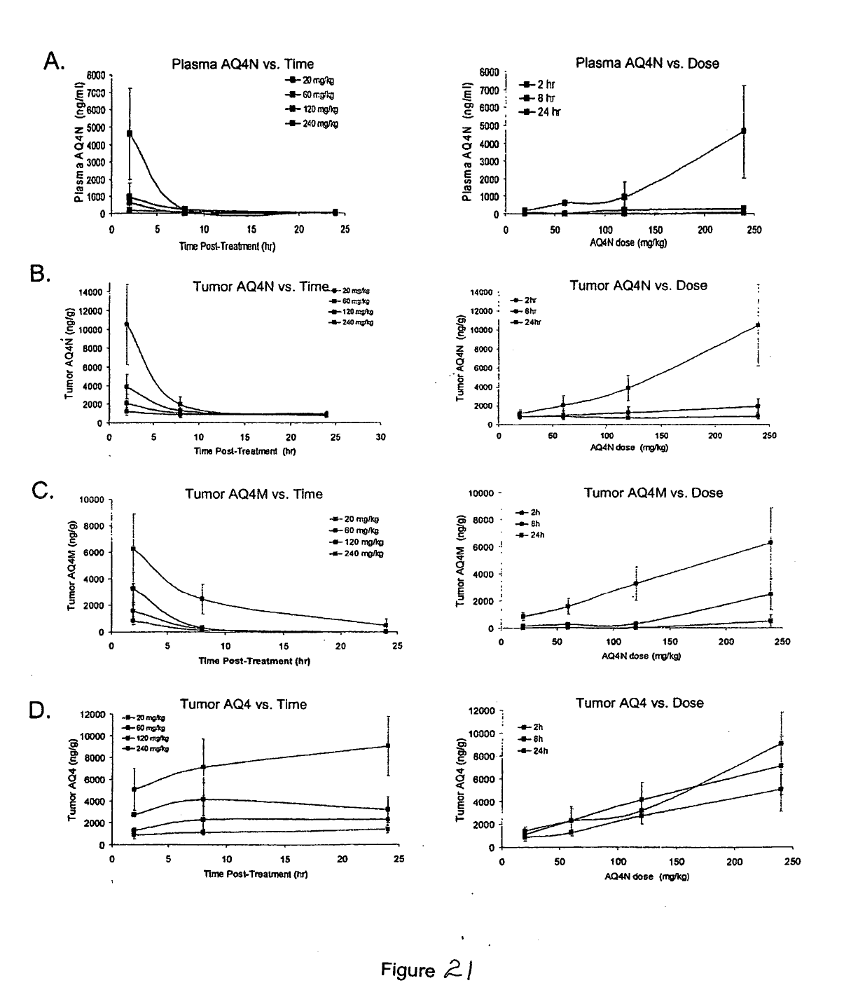

Moreover, as shown in FIG. 20A, single-agent treatment with either AQ4N or gemcitabine resulted in significant TGI in comparison to vehicle-treated groups (p<0.001). Mice treated with AQ4N at 60 mg/kg q3d×6 demonstrated the highest TGI (58.8%) compared to the vehicle-treated control group (p<0.001) (FIG. 20A). In addition, weekly treatment using AQ4N at 120 mg/kg also demonstrated significant TGI (42.4%) in a manner comparable to gemcitabine treatment (51%).

Having observed significant activity in two different sc models, we next investigated the effects of AQ4N in prolonging the survival of tumor-bearing mice in a more clinically relevant and highly aggressive orthotopic (ot) model of BxPC-3 pancreatic carcinoma. In this model, control mice that are surgically injected with tumor cells into the pancreas parenchyma succumb to tumor burden with a median survival time of 36 days (FIG. 20B). The same dose schedules of AQ4N (60 mg/kg q3d×6 and 120 mg/kg q weekly×3) that resulted in efficacy in the sc BxPC-3 model were also evaluated in this ot model with the treatment being initiated on day 14 following tumor implantation. As shown in FIG. 20B, AQ4N treatment prolonged the survival of tumor-bearing mice in a statistically significant manner. Mice treated with AQ4N at 120 mg/kg q week×3 showed a prolonged median survival of 44 days (P=0.05) while those treated with AQ4N 60 mg/kg q3d×6 had a median survival time of 41 days (P=0.03) (FIG. 20B). Interestingly, gemcitabine treatment resulted in a similar therapeutic outcome as compared to AQ4N with a median survival of 40 days (P=0.04) from this highly aggressive tumor (FIG. 20B).

### EXAMPLE 7

### Selective Tumor-Targeting of the Prodrug AQ4N in Pancreatic Xenografts In Vivo

AQ4N is designed to be a prodrug that is inert in most tissues until it is bioactivated to a potent cytotoxin under hypoxic conditions. Although tumor hypoxia, produced by physical or chemical methods, has been shown to enhance AQ4N activity against syngeneic tumors in vivo (see Wilson W R, Denny W A, Pullen S M et al., Br. J. Cancer Suppl; 27:S43-S47 (1996); and Patterson L H et al., Br. J. Cancer; 82:1984-90 (2000)), the selective tumor-targeting and pharmacokinetic properties of AQ4N and its metabolites in experimental human cancer models has not been studied extensively. Therefore, we evaluated the systemic and local tumor concentrations of the AQ4N prodrug and its bioreduced metabolites, AQ4M and AQ4, following treatment in BxPC-3-tumor-bearing mice using quantitative analytical procedures (HPLC/MS/MS).

As shown in FIG. 21, tumor and plasma pharmacokinetics of AQ4N, AQ4M and AQ4 were evaluated at 2, 8 and 24 hrs following a single iv administration of AQ4N at 20, 60, 120 or 240 mg/kg. AQ4N was only detectable in plasma samples at 2 hr following drug administration. Consistent with previous reports, AQ4N was observed to be rapidly eliminated from the systemic circulation, with only trace amounts detectable in the plasma after 24 hr at the highest dose (FIG. 21A) (see Loadman P. M. et al., Drug Metab. Dispos., 29:422-6(2001)). Importantly, the activated cytotoxic metabolite, AQ4, was not detectable in the plasma at any dose or time point, and only trace amounts of the mono-N-oxide intermediate, AQ4M, were observed at 2 hr for the 240 mg/kg dose sample. These data indicate that AQ4N does not undergo systemic bioactivation and the prodrug is rapidly eliminated from the general circulation in vivo.

Consistent with its high tissue permeability, AQ4N was found at high levels in tumors only at the earliest time point (2 hr) for all doses, demonstrating rapid penetration of the prodrug into tumor tissues. Tumor levels of AQ4N were roughly linear with dose when measured at 2 hr, with no evidence of saturation at the highest dose used (FIG. 21B). Concentrations of AQ4N detected in the tumor rapidly decreased over time, with little remaining at 24 hr for all doses (FIG. 21B). Tumor accumulation of the mono-N-oxide, AQ4M, was approximately linear with increasing dose at 2 hr following drug infusion (FIG. 21C). However, AQ4M was relatively short-lived in tumors following treatment, as expected for an intermediate metabolite, and was present at minimal levels by 8 h for most doses (FIG. 21C). For the highest treatment dose of 240 mg/kg, AQ4M tumor clearance was slower as evident by the increased AUC, but reached substantially low levels by 24 hr (FIG. 21C).

The activated cytotoxic metabolite, AQ4, was found at high levels in all tumor samples at all time points, demonstrating unambiguous localized activation of the prodrug in tumor tissues (FIG. 21D). Following treatment, AQ4 levels accumulated in the tumor in prodigious amounts (1.3-9.0 μg per gram of tumor tissue) and with rapid kinetics, as observed by 55-85% of near maximal levels after 2 hr post-infusion for all doses, and 80-100% of conversion occurring by 8 hr (FIG. 21D). The levels of AQ4 observed at 24 hr are likely to be near maximal, as little AQ4N or AQ4M appeared available at this time point for further metabolic conversion (FIG. 21B and 21C). Previous studies have demonstrated that AQ4 can be detected in tumor tissues for at least 2 weeks following a single 20 mg/kg dose of AQ4N, indicating the persistence of this highly-stable DNA intercalator in vivo. In this study, quantitative detection using HPLC/MS/MS indicate that AQ4 selectively accumulates in human BxPC-3 tumors in a dose-dependent fashion with tumors not achieving saturation following administration of the highest dose of 240 mg/kg (FIG. 21D). These data provide clear demonstration that AQ4N undergoes rapid and selective conversion into the potent anti-neoplastic metabolite, AQ4, in pancreatic tumors in vivo and supports the further development of hypoxia-activated prodrugs as tumor-targeting agents.

### EXAMPLE 8

### Effects of AQ4N Treatment in Reducing Metastasis of Orthotopic BXPC-3 Tumors

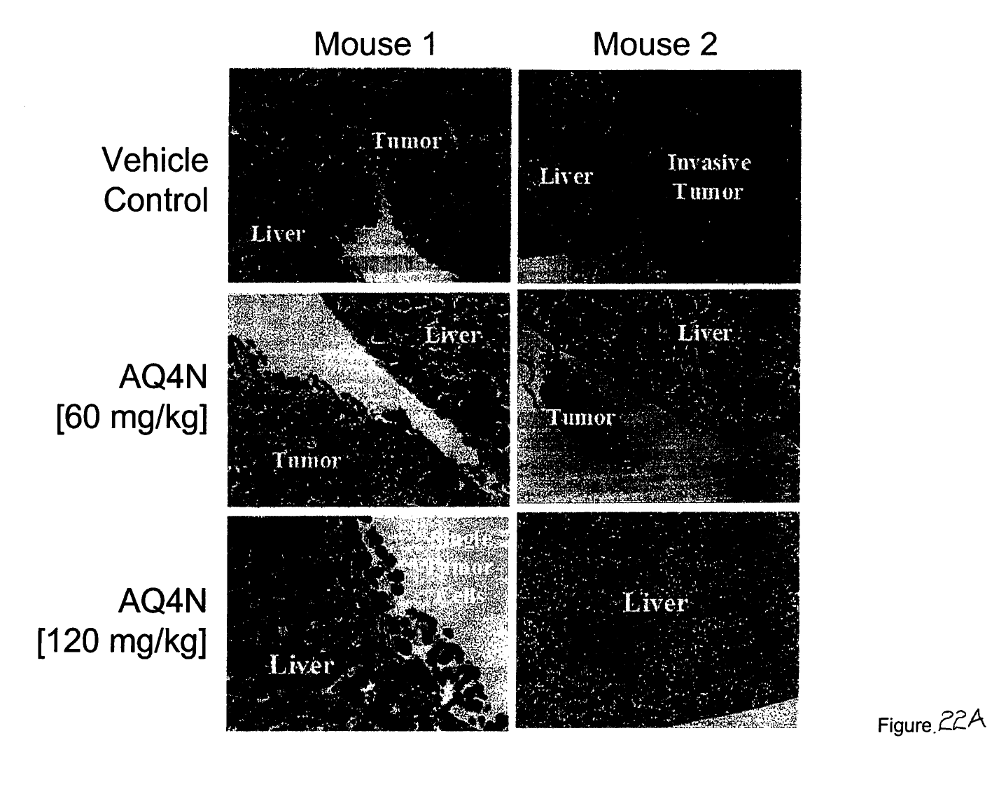

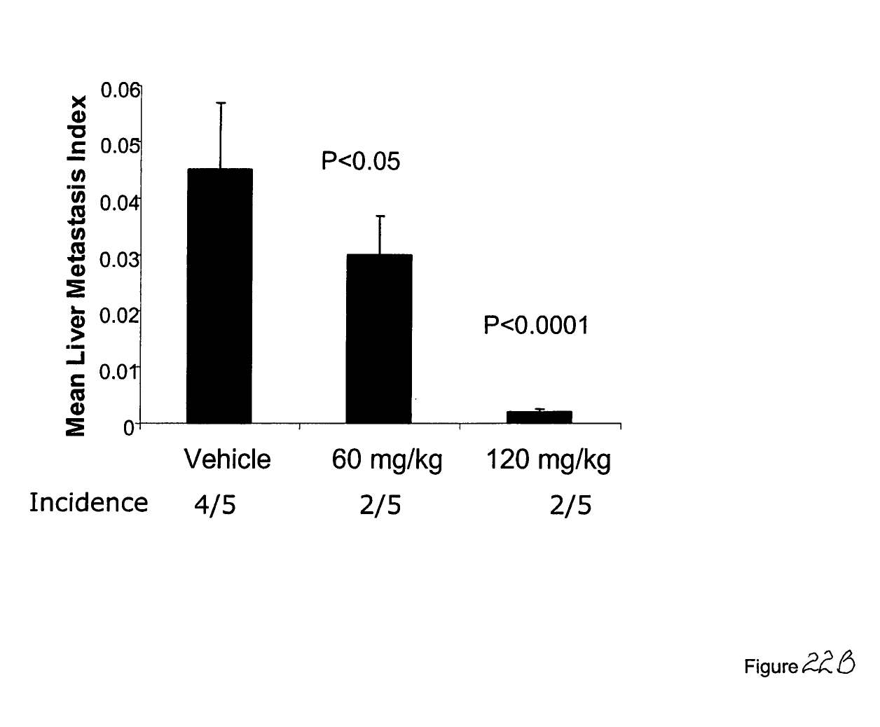

Orthotopic implantation of BxPC-3 tumor cells into the pancreas of mice results in the development of metastatic lesions in multiple organs including the liver, lymph nodes and spleen as detected by intravital microscopy (see Bouvet M. et al., Cancer Res.; 62:1534-40 (2002)). To evaluate whether AQ4N treatment had an impact on liver metastasis in this model, we performed histopathological analyses on primary liver tissue sections (n=5/group) harvested on day 36 post-tumor challenge (FIG. 22). As expected, gross pathological analysis of vehicle-control mice showed large invasive primary tumors at the site of the pancreas. In addition, histological analysis of livers from 4/5 control mice showed the presence of large, invasive tumors; an example is seen in FIG. 22A in which a metastatic lesion of approximately 50% of the size of the primary tumor is seen invading the liver. In contrast, liver metastases were only observed in only 2/5 mice following AQ4N treatment (FIG. 22B). In addition to impeding the incidence of metastatic spread, AQ4N treatment (120 mg/kg) was also observed to significantly impact the size of the invading metastatic lesion (FIG. 22A and 22B). The fraction of liver tissues occupied by metastatic tumor cells in comparison to the percentage of total liver tissue indicated a pronounced reduction in the size and percentage of metastatic tumors following AQ4N treatment (FIG. 22B). Although a dose-response was not evaluated, the anti-metastatic effects were qualitatively and quantitatively more pronounced in the 120 mg/kg AQ4N treated group than the 60 mg/kg treated group (FIG. 22A and 22B). Thus, in addition to having significant effects in delaying tumor growth progression and prolonging survival of tumor-bearing mice, AQ4N treatment also appears to have an impact in reducing liver metastasis emanating from orthotopically-implanted BxPC-3 pancreatic tumors.

### EXAMPLE 9

### AQ4N in Combination with Temozolomide: In Vitro Test

The effect of the simultaneous administration of AQ4 and temozolomide and ionising radiation to MO59K cells was examined by colony formation assay, in order to determine if these treatments combined in an additive, synergistic or antagonistic manner.

### Experimental Method

MO59K cells (glioblastoma) were dosed for 48 h with temozolomide (TMZ) (0-4 ug/ml) and AQ4 (0-2.5 ng/ml) in triplicate wells. Colonies were allowed to grow for 10 days, then stained with Giemsa stain and counted. In some experiments, the sequence of administration was varied, and in some experiments, 1.5 Gy radiation was also administered to all treatments. Note that radiation levels were not varied in these experiments. Previous data have demonstrated additive interactions between AQ4 and radiation in bladder cancer cell lines.

Note that mismatch repair-deficient cells and MGMT-overexpressing cells are known to be highly resistant to temozolomide (and other alkylating agents)—MO59K cells were chosen for this study as they are mismatch repair positive, and MGMT wild type so these mechanisms should not affect the interpretation of the results.

### Data Analysis

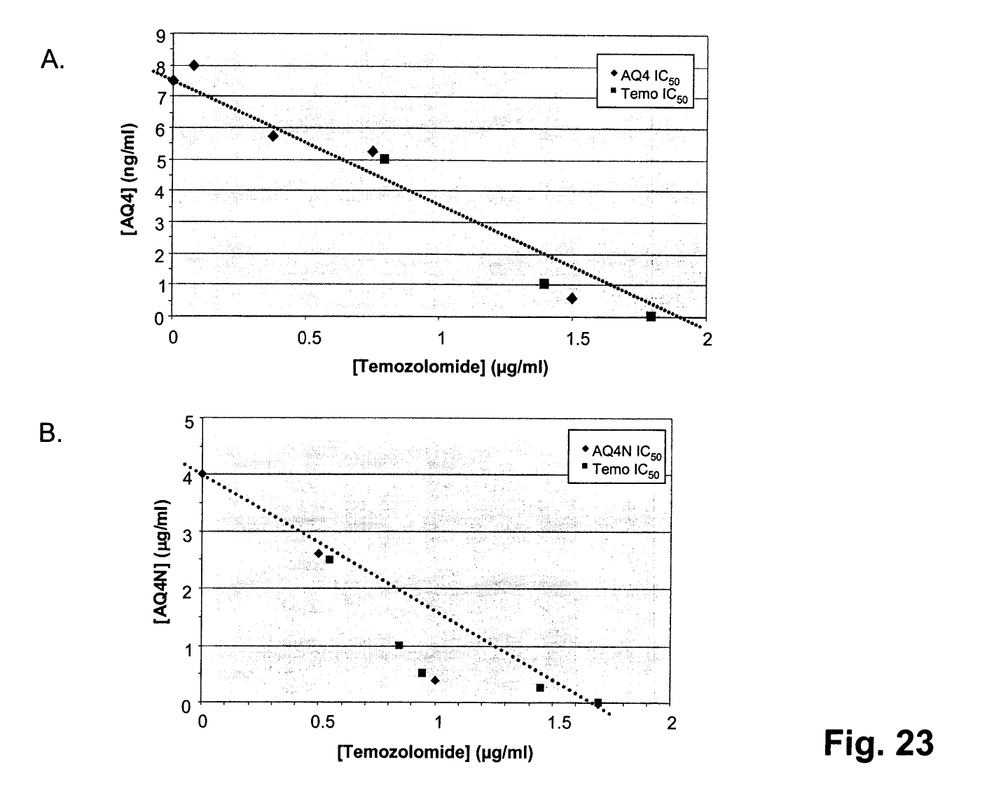

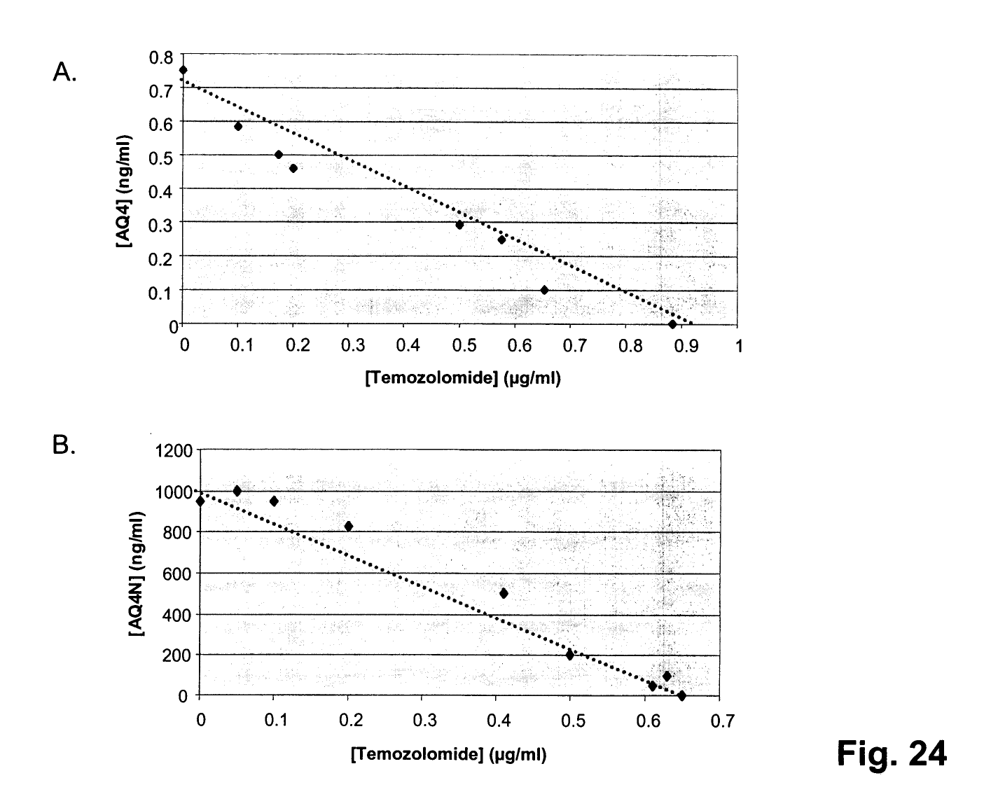

Survival curves relative to untreated controls were plotted and IC50s calculated for each combination of drug concentration (where possible). IC50 values of temozolomide at different concentrations of AQ4, and vice versa, were plotted on isobolograms as shown FIGS. 23 and 24. Points on the line are indicative of additive combinations of the two drugs, points below the line are indicative of synergistic interactions between the drugs.

### Combinations and Sequences Tested

1) AQ4 1 h, then TMZ 48 h
2) AQ4N 1 h, then TMZ 48 h
3) AQ4 1 h, then TMZ 48 h, 1 Gy radiation administered at 24 h
4) AQ4 and TMZ together 48 h, 1.5 Gy radiation administered at 24 h
5) AQ4N and TMZ together 48 h, 1.5 Gy radiation administered at 24 h

In all cases, isobolograms indicated approximate additivity of interactions, with a suggestion of potential weak synergy of AQ4N with high concentrations of TMZ.

### EXAMPLE 10

### AQ4N in Combination with Temozolomide (Clinical Test)

AQ4N is provided as a sterile solution in a single-use 5-cc glass vial with each vial containing 100 mg of AQ4N as a 30 mg/mL solution. The drug is given by IV administration in 0.9% sodium chloride infusion over approximately 30 minutes with an infusion pump.

### AQ4N Dosing: Treatment Arm

Subjects receive an AQ4N dose of 200 mg/m², 450 mg/m², or 750 mg/m² depending on the cohort they are enrolled in AQ4N is administered weekly 2-3 days prior to each week of fractionated radiotherapy (RT) for a total of six doses. Treatment Arm subjects begin focal RT within 2-3 days after beginning AQ4N, and within 35 days (5 weeks) of surgery or, if surgery cannot be performed, the biopsy that confirms the diagnosis of Glioblastoma Multiforme (GBM). Radiotherapy is given by external beam to a partial brain field in daily fractions of 2.0 Gy given 5 days per week for 6 weeks for a total planned dose of 60.0 Gy.

### Dosing: Control Arm

Subjects in the Control Arm begin focal RT within 35 days (5 weeks) of surgery or, if surgery cannot be performed, the biopsy that confirms the diagnosis of GBM. Radiotherapy is given by external beam to a partial brain field in daily fractions of 2.0 Gy given 5 days per week for 6 weeks for a total planned dose of 60.0 Gy.

### Temozolomide Dosing (Treatment and Control Arms)

During RT: Subjects in both Treatment and Control Arms are given temozolomide 75 mg/m²/day orally each day during the 6 weeks of RT, starting on the first day of RT until the last day of RT.

Post RT Adjuvant Temozolomide Dosing: Subjects in both Treatment and Control Arms is given adjuvant temozolomide (150-200 mg/m² administered on days 1-5 of a 28-day cycle), beginning 4 weeks after the end of RT. This adjuvant temozolomide schedule continues without interruption for 6 cycles, as long as there is no disease progression or toxicity due to bone marrow suppression.

The first dose of adjuvant temozolomide for the first cycle is 150 mg/m²/day. If 150 mg/m²/day dose is tolerated, the dose of temozolomide for subsequent cycles may be increased to 200 mg/m²/day.

### Duration of Treatment

Treatment with AQ4N begins 2-3 days prior to the start of RT and continues weekly for a maximum of 6 doses, or until the subject experiences an unacceptable toxicity during this six week period.

### Criteria for Evaluation

Disease progression is defined as a 25% or greater increase in the size of the product of the largest perpendicular diameters of contrast-enhancing tumor or any new tumor on CT. Disease progression is evaluated as measured by CT or MRI scans (with contrast) per standard of care.

Safety is evaluated by incidence, severity, expectedness and relatedness of clinical adverse events. Toxicity is assessed according to the NCI-CTCAE v 3.0.

It is expected that addition of AQ4N to radiotherapy and temozolomide for GBM will result in at least one of a reduction in tumor size, absence of new tumors, increase in incidence of progression-free survival (PFS), time to progression (TTP), and duration of overall survival (OS) compared to when just radiotherapy and temozolomide are administered.

Having now fully described this invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A method of treating, ameliorating, or preventing cancer comprising administering to an animal in need thereof a therapeutically effective amount of a compound of Formula I: 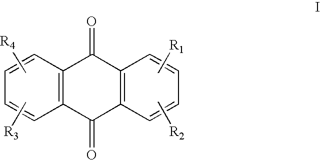
or a pharmaceutically acceptable salt or prodrug thereof, wherein:
R₁, R₂, R₃ and R₄ are independently hydrogen, hydroxy, halo, amino, C₁₋₄ alkoxy, C₂₋₈ alkanoyloxy, NH-A-NHR, or NH-A-N(O)R′R″;
A is a C₂₋₄ alkylene group with a chain length between NH and NHR or N(O)R′R″ of at least 2 carbon atoms; and
R, R′ and R″ are independently C₁₋₄ alkyl, C₂₋₄ hydroxyalkyl, or C₂₋₄ dihydroxyalkyl in which the carbon atom attached to the nitrogen atom does not carry a hydroxy group and no carbon atom is substituted by two hydroxy groups; or
R′ and R″ together are a C₂₋₆ alkylene group which with the nitrogen atom to which R′ and R″ are attached forms a heterocyclic group having 3 to 7 atoms in the ring;
with the proviso that at least one of R₁ to R₄ is NH-A-N(O)R′R″.

2. The method of claim 1, wherein said compound of Formula I is AQ4N: 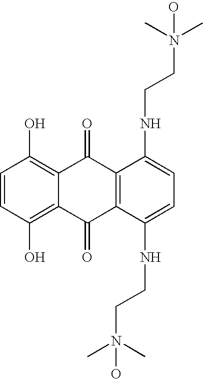
or a pharmaceutically acceptable salt or prodrug thereof.

3. The method of claim 1, wherein the cancer is colon cancer, brain cancer, glioma, multiple myeloma, head and neck cancer (except for esophageal cancer), hepatocellular cancer, melanoma, ovarian cancer, cervical cancer, renal cancer, and non-small cell lung cancer.

4. The method of claim 1, further comprising administering one or more other active agents or treatments to the animal.

5. The method of claim 4, wherein said one or more other active agents or treatments are independently selected from the group consisting of a chemotherapeutic agent and a radiotherapeutic agent/treatment.

6. The method of claim 5, wherein both one or more chemotherapeutic agents and one or more radiotherapeutic agents/treatments are administered.

7. The method of claim 5, wherein the chemotherapeutic agent is selected from the group consisting of abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

8. The method of claim 5, wherein said chemotherapeutic agent is gemcitabine or irinotecan.

9. The method of claim 5, wherein said chemotherapeutic agent is temozolomide, or a pharmaceutically acceptable salt or prodrug thereof.

10. The method of claim 4, wherein said compound having Formula I is administered prior to the administration of said active agents or treatments.

11. The method of claim 4, wherein said compound having Formula I is administered concurrently with the administration of said active agents or treatments.

12. The method of claim 11, wherein the administration of said compound having Formula I is continued beyond the administration of said active agents or treatments.

13. The method of claim 4, wherein said compound having Formula I is administered after the administration of said active agents or treatments.

14. The method of claim 4, wherein the method is repeated at least once.

15. A method of treating, ameliorating, or preventing cancer comprising administering to an animal in need thereof a radiotherapeutic agent or treatment, a therapeutically effective amount of temozolomide and a therapeutically effective amount of a compound of Formula I: 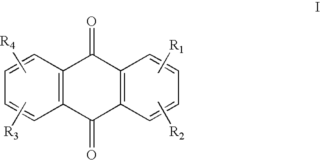
or a pharmaceutically acceptable salt or prodrug thereof, wherein:
R₁, R₂, R₃ and R₄ are independently hydrogen, hydroxy, halo, amino, C₁₋₄ alkoxy, C₂₋₈ alkanoyloxy, NH-A-NHR, or NH-A-N(O)R′R″;
A is a C₂₋₄ alkylene group with a chain length between NH and NHR or N(O)R′R″ of at least 2 carbon atoms; and
R, R′ and R″ are independently C₁₋₄ alkyl, C₂₋₄ hydroxyalkyl, or C₂₋₄ dihydroxyalkyl in which the carbon atom attached to the nitrogen atom does not carry a hydroxy group and no carbon atom is substituted by two hydroxy groups; or
R′ and R″ together are a C₂₋₆ alkylene group which with the nitrogen atom to which R′ and R″ are attached forms a heterocyclic group having 3 to 7 atoms in the ring;
with the proviso that at least one of R₁ to R₄ is NH-A-N(O)R′R″.

16. The method of claim 15 wherein the compound of formula I is AQ4N, or a pharmaceutically acceptable salt or prodrug thereof.

17. The method of claim 16, wherein the method comprises:
(a) administering 200 mg/m² to 750 mg/m² AQ4N weekly 2-3 days prior to administering fractioned radiotherapy each week;
(b) administering fractioned radiotherapy in daily fractions of 2 Gy given 5 days per week; and
(c) administering temozolomide 75 mg/m²/day daily beginning the first day of radiotherapy.

18. The method of claim 17, wherein each of AQ4N, radiotherapy, and temozolomide is administered for 6 weeks.

19. The method of claim 17, where temozolomide is administered 150 mg/m²/day to 200 mg/m²/day on days 1-5 of a 28-day cycle, beginning 4 weeks after the end radiotherapy.

20. A method of reducing or preventing metastasis of a cancer in an animal comprising administering to an animal in need thereof a therapeutically effective amount of a compound of Formula I: 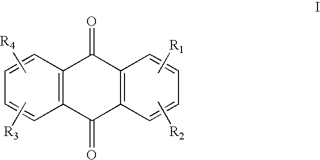
or a pharmaceutically acceptable salt or prodrug thereof, wherein:
R₁, R₂, R₃ and R₄ are independently hydrogen, hydroxy, halo, amino, C₁₋₄ alkoxy, C₂₋₈ alkanoyloxy, NH-A-NHR, or NH-A-N(O)R′R″;
A is a C₂₋₄ alkylene group with a chain length between NH and NHR or N(O)R′R″ of at least 2 carbon atoms; and
R, R′ and R″ are independently C₁₋₄ alkyl, C₂₋₄ hydroxyalkyl, or C₂₋₄ dihydroxyalkyl in which the carbon atom attached to the nitrogen atom does not carry a hydroxy group and no carbon atom is substituted by two hydroxy groups; or
R′ and R″ together are a C₂₋₆ alkylene group which with the nitrogen atom to which R′ and R″ are attached forms a heterocyclic group having 3 to 7 atoms in the ring;
with the proviso that at least one of R₁ to R₄ is NH-A-N(O)R′R″.

21. The method of claim 20 wherein the compound of formula I is AQ4N, or a pharmaceutically acceptable salt or prodrug thereof.

22. The method of claim 20, further comprising administering one or more other active agents or treatments to the animal.

23. The method of claim 22, wherein said one or more other active agents or treatments are independently selected from the group consisting of a chemotherapeutic agent and a radiotherapeutic agent/treatment.

24. The method of claim 23, wherein both one or more chemotherapeutic agents and one or more radiotherapeutic agents/treatments are administered.

25. The method of claim 23, wherein the chemotherapeutic agent is selected from the group consisting of abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

26. A method of improving the efficacy of the cytotoxic response, time to progression, or the overall survival rate of an animal receiving one or more chemotherapeutic agents and/or one or more radiotherapeutic agents/treatments comprising administering to an animal in need thereof a therapeutically effective amount of compound of Formula I: 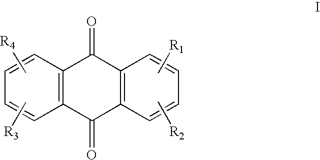
or a pharmaceutically acceptable salt or prodrug thereof, wherein:
R₁, R₂, R₃ and R₄ are independently hydrogen, hydroxy, halo, amino, C₁₋₄ alkoxy, C₂₋₈ alkanoyloxy, NH-A-NHR, or NH-A-N(O)R′R″;
A is a C₂₋₄ alkylene group with a chain length between NH and NHR or N(O)R′R″ of at least 2 carbon atoms; and
R, R′ and R″ are independently C₁₋₄ alkyl, C₂₋₄ hydroxyalkyl, or C₂₋₄ dihydroxyalkyl in which the carbon atom attached to the nitrogen atom does not carry a hydroxy group and no carbon atom is substituted by two hydroxy groups; or
R′ and R″ together are a C₂₋₆ alkylene group which with the nitrogen atom to which R′ and R″ are attached forms a heterocyclic group having 3 to 7 atoms in the ring;
with the proviso that at least one of R₁ to R₄ is NH-A-N(O)R′R″,
in combination with said one or more chemotherapeutic agents, and/or one or more radiotherapeutic agents/treatments.

27. The method of claim 26 wherein the compound of formula I is AQ4N or a pharmaceutically acceptable salt or prodrug thereof.

28. The method of claim 26, wherein both one or more chemotherapeutic agents and one or more radiotherapeutic agents/treatments are administered.

29. The method of claim 26, wherein the chemotherapeutic agent is selected from the group consisting of abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

30. A pharmaceutical composition comprising a compound of Formula I: 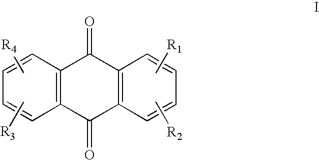
or a pharmaceutically acceptable salt or prodrug thereof, wherein:
R₁, R₂, R₃ and R₄ are independently hydrogen, hydroxy, halo, amino, C₁₋₄ alkoxy, C₂₋₈ alkanoyloxy, NH-A-NHR, or NH-A-N(O)R′R″;
A is a C₂₋₄ alkylene group with a chain length between NH and NHR or N(O)R′R″ of at least 2 carbon atoms; and
R, R′ and R″ are independently C₁₋₄ alkyl, C₂₋₄ hydroxyalkyl, or C₂₋₄ dihydroxyalkyl in which the carbon atom attached to the nitrogen atom does not carry a hydroxy group and no carbon atom is substituted by two hydroxy groups; or
R′ and R″ together are a C₂₋₆ alkylene group which with the nitrogen atom to which R′ and R″ are attached forms a heterocyclic group having 3 to 7 atoms in the ring;
with the proviso that at least one of R₁ to R₄ is NH-A-N(O)R′R″;
and one or more other chemotherapeutic agents.

31. The pharmaceutical composition of claim 30, wherein said compound of Formula I is AQ4N (compound 1): 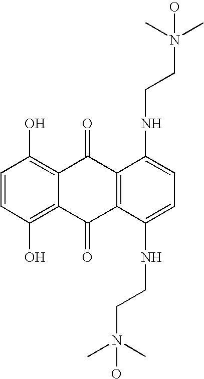
or a pharmaceutically acceptable salt or prodrug thereof.

32. The pharmaceutical composition of claim 30, wherein said chemotherapeutic agent is selected from the group consisting of abarelix, aldesleukin, alemtuzumab, alitretinoin, allopurinol, altretamine, amifostine, anastrozole, arsenic trioxide, asparaginase, BCG live, bevaceizumab, bexarotene, bleomycin, bortezomib, busulfan, calusterone, camptothecin, capecitabine, carboplatin, carmustine, celecoxib, cetuximab, chlorambucil, cinacalcet, cisplatin, cladribine, cyclophosphamide, cytarabine, dacarbazine, dactinomycin, darbepoetin alfa, daunorubicin, denileukin diftitox, dexrazoxane, docetaxel, doxorubicin, dromostanolone, Elliott's B solution, epirubicin, epoetin alfa, estramustine, etoposide, exemestane, filgrastim, floxuridine, fludarabine, fluorouracil, fulvestrant, gemcitabine, gemtuzumab ozogamicin, gefitinib, goserelin, hydroxyurea, ibritumomab tiuxetan, idarubicin, ifosfamide, imatinib, interferon alfa-2a, interferon alfa-2b, irinotecan, letrozole, leucovorin, levamisole, lomustine, meclorethamine, megestrol, melphalan, mercaptopurine, mesna, methotrexate, methoxsalen, methylprednisolone, mitomycin C, mitotane, mitoxantrone, nandrolone, nofetumomab, oblimersen, oprelvekin, oxaliplatin, paclitaxel, pamidronate, pegademase, pegaspargase, pegfilgrastim, pemetrexed, pentostatin, pipobroman, plicamycin, polifeprosan, porfimer, procarbazine, quinacrine, rasburicase, rituximab, sargramostim, streptozocin, talc, tamoxifen, tarceva, temozolomide, teniposide, testolactone, thioguanine, thiotepa, topotecan, toremifene, tositumomab, trastuzumab, tretinoin, uracil mustard, valrubicin, vinblastine, vincristine, vinorelbine, and zoledronate.

33. The composition of claim 30, wherein said chemotherapeutic agent is temozolomide.

34. The composition of claim 30, wherein said chemotherapeutic agent is gemcitabine or irinotecan.
